SEQUENCE LISTING (16 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 21 aa, Bos taurus
GIVEQCCASVCSLYQLENYCN

SEQ ID NO: 4 - protein, 30 aa, Bos taurus
FVNQHLCGSHLVEALYLVCGERGFFYTPKA

SEQ ID NO: 5 - protein, 21 aa, Sus scrofa
GIVEQCCTSICSLYQLENYCN

SEQ ID NO: 6 - protein, 30 aa, Sus scrofa; identical to SEQ ID NO: 4

SEQ ID NO: 7 - protein, 21 aa, Mus musculus
GIVDQCCTSICSLYQLENYCN

SEQ ID NO: 8 - protein, 30 aa, Mus musculus
FVKQHLCGPHLVEALYLVCGERGFFYTPKS

SEQ ID NO: 9 - DNA, 63 bp, Homo sapiens
ggcattgtggaacagtgctgcaccagcatttgcagcctgtatcagctggaaaactattgcaac

SEQ ID NO: 10 - DNA, 90 bp, Homo sapiens
tttgtgaaccagcatctgtgcggcagccatctggtggaagcgctgtatctggtgtgcggcgaacgcggctttttttataccccgaaaacc

SEQ ID NO: 11 - DNA, 63 bp, Bos taurus
ggcattgtggaacagtgctgcgcgagcgtgtgcagcctgtatcagctggaaaactattgcaac

SEQ ID NO: 12 - DNA, 90 bp, Bos taurus
tttgtgaaccagcatctgtgcggcagccatctggtggaagcgctgtatctggtgtgcggcgaacgcggctttttttataccccgaaagcg

SEQ ID NO: 13 - DNA, 63 bp, Sus scrofa; identical to SEQ ID NO: 9

SEQ ID NO: 14 - DNA, 90 bp, Sus scrofa; identical to SEQ ID NO: 12

SEQ ID NO: 15 - DNA, 63 bp, Mus musculus
ggcattgtggatcagtgctgcaccagcatttgcagcctgtatcagctggaaaactattgcaac

SEQ ID NO: 16 - DNA, 90 bp, Mus musculus
tttgtgaaacagcatctgtgcggcccgcatctggtggaagcgctgtatctggtgtgcggcgaacgcggctttttttataccccgaaaagc

(19) United States
(12) Patent Application Publication
(10) Pub. No.: US 2017/0168073 A1
(43) Pub. Date: Jun. 15, 2017

(54) CHALCONE-UREA DERIVATIVES AS INSULIN SENSORS

(71) Applicants: Agency for Science, Technology and Research, Singapore (SG); National University of Singapore, Singapore (SG)

(72) Inventors: Young-Tae CHANG, Singapore (SG); Duanting ZHAI, Singapore (SG); Sung-Chan LEE, Singapore (SG); Nam-Young KANG, Singapore (SG)

(21) Appl. No.: 15/039,150

(22) PCT Filed: Nov. 26, 2014
(86) PCT No.: PCT/SG14/00557

§ 371 Date: May 25, 2016

(30) Foreign Application Priority Data
Nov. 26, 2013 (SG) 2013087689

(51) Int. Cl. G01N 33/74; G01N 33/58; C07C 275/40; C09K 11/06; C07D 295/112

(52) U.S. Cl. CPC G01N 33/74; C09K 11/06; C07D 295/112; C07C 275/40; G01N 33/582; C09K 2211/1029; C09K 2211/1014; C09K 2211/1007; G01N 2333/62; G01N 2500/10

(57) ABSTRACT

The invention relates to compounds of formula (I) or derivatives, tautomeric forms, stereoisomers, polymorphs, hydrates, solvates, pharmaceutically acceptable salts, pharmaceutical compositions, metabolites or prodrugs thereof, in which the substituents are as defined in the specification. These compounds may be useful as insulin sensors; in insulin secretion studies and as probes for insulin detection.

### TECHNICAL FIELD

The present invention generally relates to compounds which may be useful as insulin sensors. The disclosed compounds may be useful in insulin secretion studies and probes for insulin detection.

### BACKGROUND

Diabetes has become a major global problem in the last decade with more than 336 million type 2 diabetes patients and being responsible for 4.6 million deaths each year. Diabetes is a serious healthcare problem not only because of the symptoms arising from high blood glucose levels in a patient, but also because other diseases arise from it, for example, kidney failure, myocardial infarction, stroke, blindness, and peripheral neuropathy. The direct cause of diabetes is insulin deficiency. Regulation of insulin release is essential for maintaining proper glucose concentration in blood. The detection of insulin level is crucial in the diagnosis of different types of diabetes and related disorders. The ability to detect insulin rapidly and directly is crucial in studying diabetes mellitus and pancreatic islet metabolism.

Known ways to study insulin secretion include the standard static or perifution test, in which the amount of secreted insulin in media is determined for a period of time. The standard static test is used to quantify the insulin secreting capability of islets, while the perifution test determines the rate of insulin release. Both of these techniques require ex situ analysis of insulin amount in collected samples, which is mostly done by enzyme-linked immunosorbent assay (ELISA), radioimmunoassay or chromatography. While these methods can have very low detection limits, they disadvantageously require the labelling of insulin with expensive antibodies, fluorescent tags or radioisotopes. These techniques are further disadvantageously complicated which require time consuming preparation steps and highly trained personnel. As the standard static and perifusion methods test sample fractions, the true dynamic insulin release profile is also disadvantageously beyond observation due to time averaging. Other methods utilizing electrochemistry and fluorescent gels have also been developed to detect insulin. However, their detection limits are disadvantageously unsatisfactory.

Fluorescent small molecules possess small sizes, low cost, large diversity and good sensitivity. Fluorescence techniques have been used to quantify islet secretion and reach real time in some cases, however, these studies primarily involve detecting calcium, zinc or other molecules which relate to insulin secretion. Insulin itself, as the first sequenced, first crystallized and first chemically synthesized “standard protein”, has been poorly explored in sensor development.

There is therefore a need to provide insulin sensors that overcome, or at least ameliorate, one or more of the disadvantages described above.

### SUMMARY

According to a first aspect, there is provided a compound of formula I

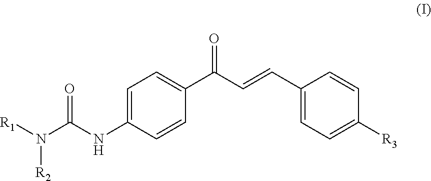

wherein the configuration around the double bond is E or Z;
wherein R₁ is selected from the group consisting of: hydrogen,
   cyclic aliphatic, acyclic aliphatic, branched aliphatic, unbranched aliphatic, substituted aliphatic, unsubstituted aliphatic,
   cyclic heteroaliphatic, acyclic heteroaliphatic, branched heteroaliphatic, unbranched heteroaliphatic, substituted heteroaliphatic, unsubstituted heteroaliphatic,
   substituted aryl, unsubstituted aryl, substituted heteroaryl, unsubstituted heteroaryl,
   cyclic acyl, acyclic acyl, substituted acyl and unsubstituted acyl;
      wherein when the group R₁ is substituted, the one or more substituents are selected from the group consisting of halogen, hydroxyl, nitro, cyano, oxo (═O), thioxo (═S), azido, nitroso, amino, hydrazine, formyl, alkyl, alkene, alkyne, -alkyalkoxy, aryl, haloalkyl, haloalkoxy, -arylalkoxy, arylalkoxy-, cycloalkyl, —O-cycloalkyl, heterocyclyl, heteroaryl, alkylamino-, -alkyleneamine, alkoxyamino-, —O—CH₂-cycloalkyl; —COOR^{a}; —C(O)R^{b}; —C(S)R^{a}; —C(O)NR^{a}R^{b}; —NR^{a}C(O)NR^{b}R^{c}; —N(R^{a})SOR^{b}; —N(R^{a})SO₂R^{b}; NR^{a}C(O)OR^{b}; —NR^{a}R^{b}; —NR^{a}C(O) R^{b}; —NR^{a}C(S)R^{b}; —SONR^{a}R^{b}; —SO₂NR^{a}R^{b}; —OR^{a}; —OR^{a}C(O)OR^{b}; —OC(O)NR^{a}R^{b}; —OC(O) R^{a}; —R^{a}NR^{b}R^{c}; —R^{a}OR^{b}; —SR^{a}; —SOR^{a} and —SO₂R^{a};
each of R^{a}, R^{b} and R^{c} are independently a hydrogen atom or an optionally substituted alkyl, aryl, arylalkyl, cycloalkyl, heterocyclyl, heteroaryl or heteroarylalkyl;
   wherein the substituents are further optionally substituted by one or more halogen, hydroxy, nitro, cycloalkyl, cyano, azido, nitroso, amino, hydrazino, formyl, alkyl, alkoxy, oxo, aryl, alkoxyamino- or haloalkyl;
wherein when the group R₁ is cyclic, R₁ is a residue formed by removing a hydrogen atom from a saturated or unsaturated monocyclic carbocyclic ring or a saturated or unsaturated monocyclic heterocyclic ring having one or two or three heteroatoms selected from a nitrogen atom, an oxygen atom and a sulfur atom in the ring, and a saturated or unsaturated monocyclic 3, 4, 5, 6, 7 or 8 membered heterocyclic ring having 3 to 5 carbon atoms, or a bicyclic or tricyclic condensed polycyclic heterocyclic ring having one or two or three heteroatoms in the ring, and a bicyclic or tricyclic condensed polycyclic heterocyclic ring having 7 to 13 carbon atoms, wherein the rings are optionally substituted with at least one substituent;
   wherein R₂ is hydrogen or alkyl;
   wherein R₃ is —NR^{a}R^{e};
each of R^{d} and R^{e} are independently selected from the group consisting of hydrogen or optionally substituted alkyl, alkenyl, aryl, arylalkyl, cycloalkyl, heterocyclyl, heteroaryl or heteroarylalkyl; or R^{d} and R^{e} together with the N, form an optionally substituted heterocyclic or heteroaryl ring;
   wherein the substituents are further optionally substituted by one or more halogen, hydroxy, nitro, cycloalkyl, cyano, azido, nitroso, amino, hydrazino, formyl, alkyl, aryl, heterocyclyl, alkoxy, mercapto, alkylthio, alkylcarbonyl, carboxy, alkxoycarbonyl, alkoxycarbonyloxy, oxo, or haloalkyl;
      wherein these further substituents are in turn further optionally substituted by one or more halogen, hydroxy, nitro, cycloalkyl, cyano, azido, nitroso, amino, hydrazino, formyl, alkyl, or haloalkyl;
         their derivatives, analogs, tautomeric forms, stereoisomers, polymorphs, hydrates, solvates, intermediates, pharmaceutically acceptable salts, pharmaceutical compositions, metabolites and prodrugs thereof.

Advantageously, the disclosed compounds may display good sensitivity and selectivity for insulin. Therefore advantageously, the disclosed compounds may be able to detect insulin rapidly and directly.

Further advantageously, the disclosed compounds may display sensitive fluorescent intensity enhancement to insulin. This may allow for fast insulin detection.

In a second aspect, there is provided a method of detecting insulin, wherein the method comprises the use of a compound defined herein.

Advantageously, the disclosed methods may allow for the fast detection of insulin.

Further advantageously, the disclosed methods may display low detection limits.

Also advantageously, the disclosed methods may not require labelling insulin with expensive antibodies, fluorescent tags or radioisotopes. Therefore, the disclosed methods may be cost-effective.

Advantageously, the disclosed methods may be simple to perform and may not require time consuming preparation steps.

Further advantageously, the disclosed methods may provide real time monitoring of insulin secretion.

In a third aspect, there is provided a compound as defined herein for use in detecting insulin.

In a fourth aspect, there is provided a use of compound as defined herein for detecting insulin.

In a fifth aspect, there is provided a method for screening a compound that is suspected to affect secretion of insulin comprised in a test sample, comprising the steps of:

a. contacting the compound to be screened with the test sample;

b. contacting a compound as defined herein with the test sample, wherein contacting is simultaneous, sequential or separate;

c. detecting a signal from a compound as defined herein, which is indicative of the presence of insulin in the test sample to obtain a first value; and

d. comparing the first value with a control value, wherein the control value corresponds to a signal in a control sample, and the control value is determined from the control sample which is the same as provided in (a), except that the control sample is not contacted with the compound to be screened;

wherein if the test value is greater or smaller than the control value, then the compound affects secretion of insulin in the sample.

In a sixth aspect, there is provided a method of assessing whether a condition or a stimulus affect secretion of insulin in cultured cells, comprising:

a. culturing cells expressing insulin under a condition or stimulus to be assessed for its effect on secretion of insulin, wherein the cells are cultured in the presence of a compound as defined herein, wherein the cells are referred to as test cells;

b. detecting a signal from the compound, which is indicative of the presence of insulin in an extracellular environment of the test cells to obtain a first value;

c. comparing the first value with a control value, wherein the control value corresponds to a signal in the extracellular environment of the control cells, and the control value is determined from control cells which are the same cells as cultured in (a), and which are cultured under the same conditions as in (a), except that the control cells are not cultured under the condition or stimulus to be assessed,

wherein if the test value is smaller or greater than the control value, then the condition or stimulus affects secretion of insulin in the cells.

Advantageously, the disclosed methods may provide real time monitoring of insulin secretion in both cultured cells and isolated islets.

Further advantageously, the disclosed methods may provide for fast insulin detection and insulin secretion study in both cultured cells and isolated islets.

### DEFINITIONS

The following are some definitions that may be helpful in understanding the description of the present invention. These are intended as general definitions and should in no way limit the scope of the present invention to those terms alone, but are put forth for a better understanding of the following description.

Unless the context requires otherwise or specifically stated to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

As used herein, the term “aliphatic” refers to an organic compound or radical characterized by a straight chain or branched chain structure, or closed ring structure, any of which may contain saturated carbon bonds, and optionally, one or more unconjugated carbon-carbon unsaturated bonds, such as a carbon-carbon double bond. For the purposes of this invention, the term “aliphatic” also includes “alicyclic” compounds defined hereinafter. The aliphatic groups may have from 1 to 24 carbon atoms eg, 1, 2, 3, 4, 5, 6, 7, 8, 9, 10, 11, 12, 13, 14, 15, 16, 17, 18, 19, 20, 21, 22, 23, 24 carbon atoms.

As used herein, the term “alkyl” includes within its meaning monovalent (“alkyl”) and divalent (“alkylene”) straight chain or branched chain saturated aliphatic groups having from 1 to 12 carbon atoms, eg, 1, 2, 3, 4, 5, 6, 7, 8, 9, 10, 11 or 12 carbon atoms. For example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, undecyl, dodecyl and the like. Alkyl groups may be optionally substituted.

As used herein, the term “alkene” includes within its meaning monovalent (“alkene” or “alkenyl”) and divalent (“alkenylene”) straight or branched chain unsaturated aliphatic hydrocarbon groups having from 2. to 10 carbon atoms, eg, 2, 3, 4, 5, 6, 7, 8, 9, or 10 carbon atoms and having at least one double bond, of either E, Z, cis or trans stereochemistry where applicable, anywhere in the alkyl chain. Examples of alkenyl groups include but are not limited to ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 1-nonenyl, 1-decenyl, and the like.

As used herein, the term “alkynyl” refers to trivalent straight chain or branched chain unsaturated aliphatic groups containing at least one carbon-carbon triple bond and having from 2 to 6 carbon atoms, eg, 2, 3, 4, 5 or 6 carbon atoms. For example, the term alkynyl includes, but is not limited to, ethynyl, propynyl, 1-butynyl, 2-butynyl, 1-pentynyl, 2-pentynyl, 1-hexynyl, 2-hexynyl, 3-hexynyl, 3-methyl-1-pentynyl, and the like. alkynyl groups may be optionally substituted.

As used herein, the term “amine” or “amino” refers to groups of the form —NR^{a}R^{b} wherein R^{a} and R^{b} are individually selected from the group including but not limited to hydrogen, optionally substituted alkyl, optionally substituted alkenyl, optionally substituted alkynyl, and optionally substituted aryl groups.

As used herein, the term “amide” refers to compounds or moieties that contain a nitrogen atom bound to the carbon of a carbonyl. The term includes “alkaminocarbonyl” or “alkylaminocarbonyl” groups which include alkyl, alkenyl, aryl or alkynyl groups bound to an amino group bound to a carbonyl group. It includes “arylaminocarbonyl” and “arylcarbonylamino” groups which include aryl or heteroaryl moieties bound to an amino group which is bound to the carbon of a carbonyl. The terms “alkylaminocarbonyl,” “alkenylaminocarbonyl,” “alkynylaminocarbonyl,” “arylaminocarbonyl,” “alkylcarbonylamino,” “alkenylcarbonylamino,” “alkynylcarbonylamino,” and “arylcarbonylamino” are included in term “amide.” Amides also include urea groups (aminocarbonylamino) and carbamates-(oxycarbonylamino).

As used herein, the term “aryl”, or variants such as “aromatic group” or “arylene” refers to monovalent (“aryl”) and divalent (“arylene”) single, polynuclear, conjugated and fused residues of aromatic hydrocarbons having from 6 to 10 carbon atoms. Such groups include, for example, phenyl, biphenyl, naphthyl, phenanthrenyl, and the like. Aryl groups may be optionally substituted.

As used herein, the term “carbocycle”, or variants such as “carbocylic” or “carbocyclic ring”, includes within its meaning any stable 3, 4, 5, 6, or 7-membered monocyclic or bicyclic or 7, 8, 9, 10, 11, 12, or 13-membered bicyclic or tricyclic, any of which may be saturated, partially unsaturated, or aromatic. Examples of such carbocycles include, but are not limited to, cyclopropyl, cyclobutyl, cyclopentyl, cyclohexyl, cycloheptyl, adamantyl, cyclooctyl, [3.3.0]bicyclooctane, [4.3.0]bicyclononane, [4.4.0]bicyclodecane (decalin), [2.2.2]bicyclooctane, fluorenyl, phenyl, naphthyl, indanyl, adamantyl, or tetrahydronaphthyl (tetralin). Preferred carbocycles, unless otherwise specified, are cyclopropyl, cyclobutyl, cyclopentyl, cyclohexyl, phenyl, naphthyl, and indanyl. When the term “carbocycle” is used, it is intended to include “aryl”. Carbocycles may be optionally substituted.

As used herein, the term “cycloalkyl” refers to a non-aromatic mono- or multicyclic ring system comprising about 3 to about 10 carbon atoms. The cycloalkyl can be optionally substituted with one or more “ring system substituents” which may be the same or different, and are as defined herein. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like. Further non-limiting examples of cycloalkyl include the following:

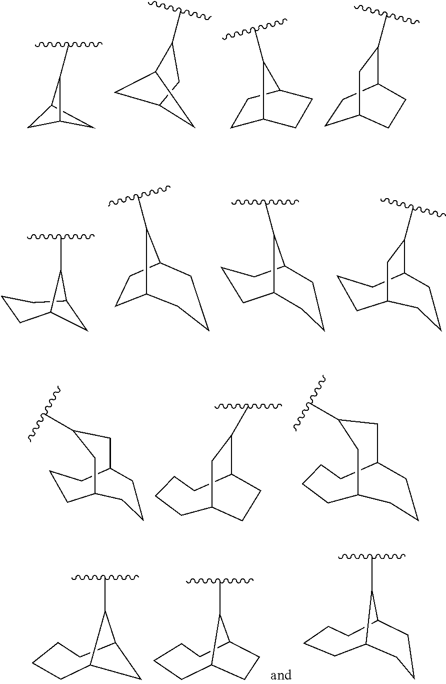

The term “cycloalkenyl” as used herein refers to a non-aromatic mono or multicyclic ring system comprising about 3 to about 10 carbon atoms which contains at least one carbon-carbon double bond. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl, as well as unsaturated moieties of the examples shown above for cycloalkyl. Cycloalkenyl groups may be optionally substituted.

The term “heteroaliphatic” as used herein refers to an aliphatic moiety as defined above, having one or more carbon atoms, for example 1, 2, 3, 4, 5, 6, 7, 8, 9, 10 carbon atoms, replaced with one or more heteroatoms, which may be the same or different, where the point of attachment to the remainder of the molecule is through a carbon atom of the heteroaliphatic radical, or the heteroatom. Suitable heteroatoms include 0, S, and N. Non-limiting examples include ethers, thioethers, amines, hydroxymethyl, 3-hydroxypropyl, 1,2-dihydroxyethyl, 2-methoxyethyl, 2-aminoethyl, 2-dimethylaminoethyl, and the like. Heteroaliphatic groups may be optionally substituted.

As used herein, the term “heteroaryl” as used herein refers to an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like. Heteroaryl groups may be optionally substituted.

As used herein, the term “heterocycle”, or variants such as “heterocylic” or “heterocyclic ring”, includes within its meaning a group comprising a covalently closed ring herein at least one atom forming the ring is a carbon atom and at least one atom forming the ring is a heteroatom. Heterocyclic rings may be formed by three, four, five, six, seven, eight, nine, or more than nine atoms, any of which may be saturated, partially unsaturated, or aromatic. Any number of those atoms may be heteroatoms (i.e., a heterocyclic ring may comprise one, two, three, four, five, six, seven, eight, nine, or more than nine heteroatoms). Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocylic ring will have additional heteroatoms in the ring. In heterocycles comprising two or more heteroatoms, those two or more heteroatoms may be the same or different from one another. Heterocycles may be optionally substituted. Binding to a heterocycle can be at a heteroatom or via a carbon atom. Examples of heterocycles include heterocycloalkyls (where the ring contains fully saturated bonds) and heterocycloalkenyls (where the ring contains one or more unsaturated bonds) such as, but are not limited to the following:

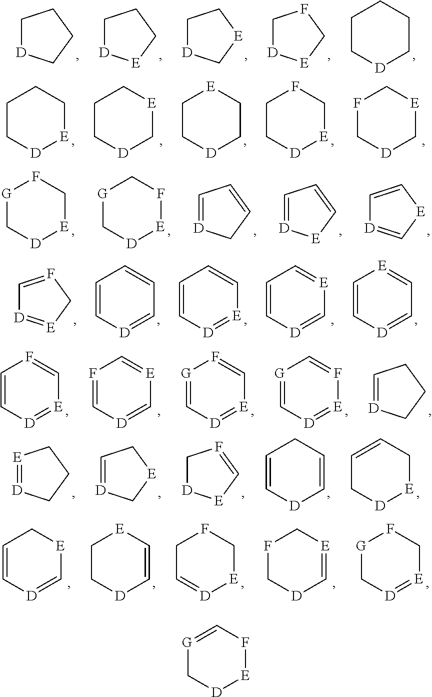

wherein D, E, F, and G independently represent a heteroatom. Each of D, E, F, and G may be the same or different from one another.

As used herein, the term “optionally substituted” means the group to which this term refers may be unsubstituted, or may be substituted with one or more groups other than hydrogen provided that the indicated atom's normal valency is not exceeded, and that the substitution results in a stable compound. Such groups may be, for example, halogen, hydroxy, oxo, cyano, nitro, alkyl, alkoxy, haloalkyl, haloalkoxy, aryl4alkoxy, alkylthio, hydroxyalkyl, alkoxyalkyl, cycloalkyl, cycloalkylalkoxy, alkanoyl, alkoxycarbonyl, alkylsulfonyl, alkylsulfonyloxy, alkylsulfonylalkyl, arylsulfonyl, arylsulfonyloxy, arylsulfonylalkyl, alkylsulfonamido, alkylamido, alkylsulfonamidoalkyl, alkylamidoalkyl, arylsulfonamido, arylcarboxamido, arylsulfonamidoalkyl, arylcarboxamidoalkyl, aroyl, aroyl4alkyl, arylalkanoyl, acyl, aryl, arylalkyl, alkylaminoalkyl, a group R^{x}R^{y}N—, R^{x}CO(CH₂)ₘ, R^{x}CON(R^{y}) (CH₂)ₘ, R^{x}R^{y}NCO(CH₂)ₘ, R^{x}R^{y}NSO₂ (CH₂)ₘ or R^{x}SO₂NR^{y} (CH₂)ₘ (where each of R^{x} and R^{y} is independently selected from hydrogen or alkyl, or where appropriate R^{x}R^{y} forms part of carbocylic or heterocyclic ring and m is 0, 1, 2, 3 or 4), a group R^{x}R^{y}N(CH₂)ₚ— or R^{x}R^{y}N(CH₂)ₚO— (wherein p is 1, 2, 3 or 4); wherein when the substituent is R^{x}R^{y}N(CH₂)ₚ— or R^{x}R^{y}N(CH₂)ₚO, R^{x} with at least one CH₂ of the (CH₂)ₚ, portion of the group may also form a carbocyclyl or heterocyclyl group and R^{y} may be hydrogen, alkyl.

As used herein, the term “substituted” means the group to which this term refers is substituted with one or more groups other than hydrogen provided that the indicated atom's normal valency is not exceeded, and that the substitution results in a stable compound. Such groups may be, for example, halogen, hydroxy, oxo, cyano, nitro, alkyl, alkoxy, haloalkyl, haloalkoxy, arylalkoxy, alkylthio, hydroxyalkyl, alkoxyalkyl, cycloalkyl, cycloalkylalkoxy, alkanoyl, alkoxycarbonyl, alkylsulfonyl, alkylsulfonyloxy, alkylsulfonylalkyl, arylsulfonyl, arylsulfonyloxy, arylsulfonylalkyl, alkylsulfonamido, alkylamido, alkylsulfonamidoalkyl, alkylamidoalkyl, arylsulfonamido, arylcarboxamido, arylsulfonamidoalkyl, arylcarboxamidoalkyl, aroyl, aroyl4alkyl, arylalkanoyl, acyl, aryl, arylalkyl, alkylaminoalkyl, a group R^{x}R^{y}N—, R^{x}OCO(CH₂)ₘ, R^{x}CON (R^{y}) (CH₂)ₘ, R^{x}R^{y}NCO (CH₂)ₘ, R^{x}R^{y}NSO₂ (CH₂)ₘ or —R^{x}SO₂NR^{y}(CH₂)ₘ (where each of R^{x} and R^{y} is independently selected from hydrogen or alkyl, or where appropriate R^{x}R^{y} forms part of carbocylic or heterocyclic ring and m is 0, 1, 2, 3 or 4), a group R^{x}R^{y}N(CH₂)ₚ— or R^{x}R^{y}N(CH₂)ₚO— (wherein p is 1, 2, 3 or 4); wherein when the substituent is R^{x}R^{y}N(CH₂)ₚ— or R^{x}R^{y}N(CH₂)ₚO, R^{x} with at least one CH₂ of the (CH₂), portion of the group may also form a carbocyclyl or heterocyclyl group and R^{y} may be hydrogen, alkyl.

Any carbon or heteroatom with unsatisfied valences in the text, schemes, examples, structural formulae, and any Tables herein is assumed to have the hydrogen atom or atoms to satisfy the valences.

When compounded chemical names, e.g. “arylalkyl” and “arylimine” are used herein, they are understood to have a specific connectivity to the core of the chemical structure. The group listed farthest to the right (e.g. alkyl in “arylalkyl”), is the group that is directly connected to the core. Thus, an “arylalkyl” group, for example, is an alkyl group substituted with an aryl group (e.g. phenylmethyl (i.e., benzyl)) and the alkyl group is attached to the core. An “alkylaryl” group is an aryl group substituted with an alkyl group (e.g., p-methylphenyl (i.e., p-tolyl)) and the aryl group is attached to the core.

As used herein, the term “derivative” refers to compounds that have a common core structure, and are substituted with various groups as described herein.

As used herein, the term “pharmaceutically acceptable salt” includes, for example, the hydrochloride, hydrobromide, hydroiodide, sulfate, phosphate, acetate, propionate, lactate, maleate, malate, succinate, and tartrate salts.

As used herein, the term “pharmaceutical composition” refers to a formulation containing the disclosed compounds in a form suitable for administration to a subject. The pharmaceutical composition can be in bulk or in unit dosage form. The unit dosage form can be in any of a variety of forms, including, for example, a capsule, an IV bag, a tablet, a single pump on an aerosol inhaler, or a vial. The quantity of active ingredient (i.e., a formulation of the disclosed compound or salts thereof) in a unit dose of composition is an effective amount and may be varied according to the particular treatment involved.

It may be appreciated that it may be necessary to make routine variations to the dosage depending on the age and condition of the patient. The dosage will also depend on the route of administration. A variety of routes are contemplated, including topical, oral, pulmonary, rectal, vaginal, parenternal, transdermal, subcutaneous, intravenous, intramuscular, intraperitoneal and intranasal. The compounds described herein, and the pharmaceutically acceptable salts thereof can be used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein. For oral administration, the disclosed compounds or salts thereof can be combined with a suitable solid or liquid carrier or diluent to form capsules, tablets, pills, powders, syrups, solutions, suspensions and the like. The tablets, pills, capsules, and the like contain from about 1 to about 99 weight percent of the active ingredient and a binder such as gum tragacanth, acacias, corn starch or gelatin; excipients such as dicalcium phosphate; a disintegrating agent such as corn starch, potato starch or alginic acid; a lubricant such as magnesium stearate; and/or a sweetening agent such as sucrose, lactose or saccharin. When a dosage unit form is a capsule, it may contain, in addition to materials of the above type, a liquid carrier such as a fatty oil. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor, and the like.

For parental administration of the disclosed compounds, or salts, solvates, or hydrates thereof, can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

In addition to the formulations previously described, the compounds may also be formulated as a depot preparation. Suitable formulations of this type include biocompatible and biodegradable polymeric hydrogel formulations using crosslinked or water insoluble polysaccharide formulations, polymerizable polyethylene oxide formulations, impregnated membranes, and the like. Such long acting formulations may be administered by implantation or transcutaneous delivery (for example subcutaneously or intramuscularly), intramuscular injection or a transdermal patch. Preferably, they are implanted in, or applied to, the microenvironment of an affected organ or tissue, for example, a membrane impregnated with the disclosed compound can be applied to an open wound or burn injury. Thus, for example, the compounds may be formulated with suitable polymeric or hydrophobic materials, for example, as an emulsion in an acceptable oil, or ion exchange resins, or as sparingly soluble derivatives, for example, as a sparingly soluble salt.

For topical administration, suitable formulations may include biocompatible oil, wax, gel, powder, polymer, or other liquid or solid carriers. Such formulations may be administered by applying directly to affected tissues, for example, a liquid formulation to treat infection of conjunctival tissue can be administered, dropwise to the subject's eye, a cream formulation can be administer to a wound site, or a bandage may be impregnated with a formulation, and the like.

For rectal administration, suitable pharmaceutical compositions are, for example, topical preparations, suppositories or enemas.

For vaginal administration, suitable pharmaceutical compositions are, for example, topical preparations, pessaries, tampons, creams, gels, pastes, foams or sprays.

In addition, the compounds may also be formulated to deliver the active agent by pulmonary administration, e.g., administration of an aerosol formulation containing the active agent from, for example, a manual pump spray, nebulizer or pressurized metered-dose inhaler. Suitable formulations of this type can also include other agents, such as antistatic agents, to maintain the disclosed compounds as effective aerosols.

As used herein, the term “prodrug” refers in one embodiment to a metabolic precursor of a disclosed compound which is pharmaceutically acceptable. A prodrug may be inactive when administered to a subject but is converted in vivo to an active compound. In one embodiment, the term “metabolite” or “active metabolite”, refers to a metabolic product of a disclosed compound that is pharmaceutically and/or pharmacologically beneficial and/or effective. Prodrugs and active metabolites may be determined using techniques known in the art. Prodrugs and active metabolites of a compound may be identified using routine techniques known in the art.

Prodrugs are often useful because, in some embodiments, they may be easier to administer or process than the parent drug. They may, for instance, be bioavailable by oral administration whereas the parent is not. The prodrug may also have improved solubility in pharmaceutical compositions over the parent drug. An embodiment of a prodrug would be an amino acid bonded to a primary hydroxyl group where the amino acid is metabolized to reveal the active moiety. In certain embodiments, upon in vivo administration, a prodrug is chemically converted to the biologically, pharmaceutically or therapeutically more active form of the compound. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. The prodrug is designed to alter the metabolism or the transport characteristics of a drug in certain embodiments, to mask side-effects or toxicity, to improve the flavor of a drug or to alter other characteristics or properties of a drug in other discrete embodiments. By virtue of knowledge of pharmacodynamic processes and drug metabolism in vivo, those of skill in this art, once a pharmaceutically active compound is known, can design prodrugs of the compound.

As used herein, the terms “metabolite” or “active metabolite” may also refer to a biologically active derivative of a compound that is formed when the compound is metabolized. The term “metabolized,” refers in one embodiment to the sum of the processes (including, but not limited to, hydrolytic reactions and reactions catalyzed by enzymes, such as, oxidation reactions, de-esterification reactions and/or proteolytic reactions) by which a particular substance is changed by an organism. Thus, enzymes may produce specific structural alterations to a compound. In one embodiment, cytochrome P450 catalyzes a variety of oxidative and reductive reactions while some isoforms, such as CYP3A4 are involved in de-esterification. Metabolites of the compounds disclosed herein can be identified either by administration of compounds to a host under conditions allowing for the determination of activity by the metabolite and analysis of tissue samples from the host, or by incubation of compounds with hepatic cells in vitro and analysis of the resulting compounds. Both methods are well known in the art. In some embodiments, a compound is metabolized to pharmacologically active metabolites.

As used herein, the term “pharmaceutically acceptable carrier” is intended to include solvents, dispersion media, coatings, anti-bacterial and anti-fungal agents, isotonic and absorption delaying agents, and the like. The use of such media and agents for pharmaceutically active substances is well known in the art. Except insofar as any conventional media or agent is incompatible with the compound, use thereof in the therapeutic compositions and methods of treatment and prophylaxis is contemplated. Supplementary active compounds may also be incorporated into the compositions according to the present invention. It is especially advantageous to formulate parenteral compositions in dosage unit form for ease of administration and uniformity of dosage. “Dosage unit form” as used herein refers to physically discrete units suited as unitary dosages for the individual to be treated; each unit containing a predetermined quantity of compound(s) is calculated to produce the desired therapeutic effect in association with the required pharmaceutical carrier. The compound(s) may be formulated for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

The present invention includes within its scope all isomeric forms of the compounds disclosed herein, including all diastereomeric isomers, racemates and enantiomers, for example, E, Z, cis, trans, (R), (S), (L), (D), (+), and/or (−) forms of the compounds, as appropriate in each case.

The word “substantially” does not exclude “completely” e.g. a composition which is “substantially free” from Y may be completely free from Y. Where necessary, the word “substantially” may be omitted from the definition of the invention.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Exemplary, non-limiting embodiments of the disclosed insulin sensors will now be disclosed.

As discussed above, the present disclosure provides an insulin sensor.

Said insulin sensor may be a compound of formula I:

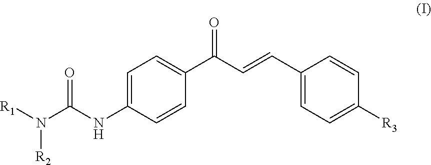

wherein the configuration around the double bond is E or Z;
wherein:
R₁ is selected from the group consisting of hydrogen, alkyl, alkenyl, alkynyl, heteroalkyl, heteroalkenyl, heteroalkynyl, carbocyclyl, cycloalkyl, cycloalkenyl, heterocyclyl, aryl, heteroaryl, or acyl, wherein any of alkyl, alkenyl, alkynyl, heteroalkyl, heteroalkenyl, heteroalkynyl, carbocyclyl, cycloalkyl, cycloalkenyl, heterocyclyl, aryl, heteroaryl or acyl is optionally substituted;
R₂ is hydrogen or alkyl;
R₃ is —NR^{d}R^{e};
each of R^{d} and R^{e} are independently selected from the group consisting of hydrogen or alkyl, alkenyl, alkynyl, aryl, arylalkyl, cycloalkyl, heterocyclyl, heteroaryl or heteroarylalkyl, wherein any of alkyl, alkenyl, alkynyl, aryl, arylalkyl, cycloalkyl, heterocyclyl, heteroaryl or heteroarylalkyl is optionally substituted; or
R^{d} and R^{e} together with the N, form an optionally substituted heterocyclic or optionally substituted heteroaryl ring;
   their derivatives, analogs, tautomeric forms, stereoisomers, polymorphs, hydrates, solvates, intermediates, pharmaceutically acceptable salts, pharmaceutical compositions, metabolites and prodrugs thereof.

In another embodiment, the insulin sensor may be a compound of formula I:

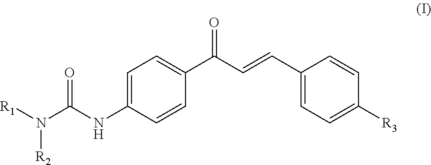

wherein the configuration around the double bond is E or Z;
wherein R₁ is selected from the group consisting of hydrogen, cyclic or acyclic, branched or unbranched, substituted or unsubstituted aliphatic, cyclic or acyclic, branched or unbranched, substituted or unsubstituted heteroaliphatic, substituted or unsubstituted aryl, substituted or unsubstituted heteroaryl, and cyclic or acyclic, substituted or unsubstituted acyl;
   when the group R₁ is substituted, the substituent is selected from the group consisting of halogens, hydroxyl, nitro, cyano, oxo (═O), thioxo (═S), azido, nitroso, amino, hydrazine, formyl, alkyl, alkene, alkylene, alkyalkoxy, aryl, haloalkyl, haloalkoxy, arylalkoxy, cycloalkyl, —O-cycloalkyl, heterocyclyl, heteroaryl, alkylamino, alkoxyamines, —O—CH₂-cycloalkyl; —COOR^{a}; —C(O)R^{b}; —C(S)R^{a}; —C(O)NR^{a}R^{b}; —NR^{a}C(O)NR^{b}R^{c}; —N(R^{a}) SOR^{b}; —N(R^{a}) SO₂R^{b}; NR^{a}C(O)OR^{b}; —NR^{a}R^{b}; —NR^{a}C(O) R^{b}; —NR^{a}C(S) R^{b}; SONR^{a}R^{b}; —SO₂NR^{a}R^{b}; —OR^{a}; —OR^{a}C(O)OR^{b}; —OC(O)NR^{a}R^{b}; —OC(O) R^{a}; —R^{a}NR^{b}R^{c}; —R^{a}OR^{b}; —SR^{a}; —SOR^{a} and —SO₂R^{a};
   R^{a}, R^{b} and R^{c} is independently a hydrogen atom or an optionally substituted alkyl, aryl, arylalkyl, cycloalkyl, heterocyclyl, heteroaryl or heteroarylalkyl;
      wherein the substituents are further optionally substituted by any one of halogens, hydroxy, nitro, cycloalkyl, cyano, azido, nitroso, amino, hydrazino, formyl, alkyl or haloalkyl;
when the group R₁ is cyclic, R₁ is a residue formed by removing a hydrogen atom from a saturated or unsaturated monocyclic carbocyclic ring or a saturated or unsaturated monocyclic heterocyclic ring having one or two or three heteroatoms selected from a nitrogen atom, an oxygen atom and a sulfur atom in the ring, and a saturated or unsaturated monocyclic 3, 4, 5, 6, 7 or 8 membered heterocyclic ring having 3 to 5 carbon atoms, or a bicyclic or tricyclic condensed polycyclic heterocyclic ring having one or two or three heteroatoms in the ring, and a bicyclic or tricyclic condensed polycyclic heterocyclic ring having 7 to 13 carbon atoms, wherein the rings are optionally substituted with at least one substituent;
wherein R₂ is hydrogen or alkyl;
wherein R₃ is —NR^{d}R^{e};
   R^{d} and R^{e} is independently selected from the group consisting of hydrogen or an optionally substituted alkyl, aryl, arylalkyl, cycloalkyl, heterocyclyl, heteroaryl or heteroarylalkyl;
      wherein the substituents are further optionally substituted by halogens, hydroxy, nitro, cycloalkyl, cyano, azido, nitroso, amino, hydrazino, formyl, alkyl, haloalkyl;
their derivatives, analogs, tautomeric forms, stereoisomers, polymorphs, hydrates, solvates, intermediates, pharmaceutically acceptable salts, pharmaceutical compositions, metabolites and prodrugs thereof.

In another embodiment, the insulin sensor may be a compound of formula I:

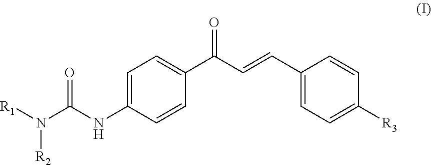

wherein the configuration around the double bond is E or Z;
wherein R₁ is selected from the group consisting of hydrogen, cyclic or acyclic, branched or unbranched, substituted or unsubstituted aliphatic, cyclic or acyclic, branched or unbranched, substituted or unsubstituted heteroaliphatic, substituted or unsubstituted aryl, substituted or unsubstituted heteroaryl, and cyclic or acyclic, substituted or unsubstituted acyl;
wherein when the group R₁ is substituted, the one or more substituents are selected from the group consisting of halogen, hydroxyl, nitro, cyano, oxo (═O), thioxo (═S), azido, nitroso, amino, hydrazine, formyl, alkyl, alkene, alkyne, -alkyalkoxy, aryl, haloalkyl, haloalkoxy, -arylalkoxy, arylalkyoxy-, cycloalkyl, —O-cycloalkyl, heterocyclyl, heteroaryl, alkylamino-, -alkyleneamine, alkoxyamino-, —O—CH₂-cycloalkyl; —COOR^{a}; —C(O)R^{b}; —C(S)R^{a}; —C(O)NR^{a}R^{b}; —NR^{a}C(O)NR^{b}R^{c}; —N(R^{a}) SOR^{b}; —N(R^{a})SO₂R^{b}; —NR^{a}C(O)OR^{b}; NR^{a}R^{b}; —NR^{a}C(O) R^{b}; —NR^{a}C(S) R^{b}; —SONR^{a}R^{b}; —SO₂NR^{a}R^{b}; —OR^{a}; —OR^{a}C(O)OR^{b}; —OC(O)NR^{a}R^{b}; —OC(O) R^{a}; —R^{a}NR^{b}R^{c}; —R^{a}OR^{b}; SR^{a}; —SOR^{a} and —SO₂R^{a};
   R^{a}, R^{b} and R^{c} is independently a hydrogen atom or an optionally substituted alkyl, aryl, arylalkyl, cycloalkyl, heterocyclyl, heteroaryl or heteroarylalkyl;
      wherein the substituents are further optionally substituted by one or more of halogens, hydroxy, nitro, cycloalkyl, cyano, azido, nitroso, amino, hydrazino, formyl, alkyl, alkoxy, oxo, aryl, alkoxyamino- or haloalkyl;
when the group R₁ is cyclic, R₁ is a residue formed by removing a hydrogen atom from a saturated or unsaturated monocyclic carbocyclic ring or a saturated or unsaturated monocyclic heterocyclic ring having one or two or three heteroatoms selected from a nitrogen atom, an oxygen atom and a sulfur atom in the ring, and a saturated or unsaturated monocyclic 3, 4, 5, 6, 7 or 8 membered heterocyclic ring having 3 to 5 carbon atoms, or a bicyclic or tricyclic condensed polycyclic heterocyclic ring having one or two or three heteroatoms in the ring, and a bicyclic or tricyclic condensed polycyclic heterocyclic ring having 7 to 13 carbon atoms, wherein the rings are optionally substituted with at least one substituent;
   wherein R₂ is hydrogen or alkyl;
   wherein R₃ is —NR^{d}R^{e};
R^{d} and R^{e} is independently selected from the group consisting of hydrogen or an optionally substituted alkyl, alkenyl, aryl, arylalkyl, cycloalkyl, heterocyclyl, heteroaryl or heteroarylalkyl; or R^{d} and R^{e} together with the N, form an optionally substituted heterocyclic or heteroaryl ring;
   wherein the substituents are further optionally substituted by one or more halogen, hydroxy, nitro, cycloalkyl, cyano, azido, nitroso, amino, hydrazino, formyl, alkyl, aryl, heterocyclyl, alkoxy, mercapto, alkylthio, alkylcarbonyl, carboxy, alkxoycarbonyl, alkoxycarbonyloxy, oxo, or haloalkyl;
   wherein these further substituents are in turn further optionally substituted by one or more halogen, hydroxy, nitro, cycloalkyl, cyano, azido, nitroso, amino, hydrazino, formyl, alkyl, haloalkyl;
      their derivatives, analogs, tautomeric forms, stereoisomers, polymorphs, hydrates, solvates, intermediates, pharmaceutically acceptable salts, pharmaceutical compositions, metabolites and prodrugs thereof.

In another embodiment, the insulin sensor may be a compound of formula I:

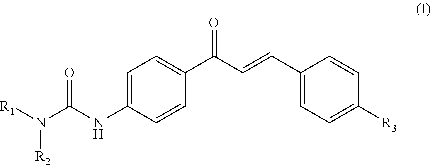

wherein the configuration around the double bond is E or Z;
wherein R₁ is selected from the group consisting of:
hydrogen,
cyclic aliphatic, acyclic aliphatic, branched aliphatic, unbranched aliphatic, substituted aliphatic, unsubstituted aliphatic,
cyclic heteroaliphatic, acyclic heteroaliphatic, branched heteroaliphatic, unbranched heteroaliphatic, substituted heteroaliphatic, unsubstituted heteroaliphatic,
substituted aryl, unsubstituted aryl,
substituted heteroaryl, unsubstituted heteroaryl,
cyclic acyl, acyclic acyl, substituted acyl and unsubstituted acyl;
   wherein when the group R₁ is substituted, the one or more substituents are selected from the group consisting of halogen, hydroxyl, nitro, cyano, oxo (═O), thioxo (═S), azido, nitroso, amino, hydrazine, formyl, alkyl, alkene, alkyne, -alkyalkoxy, aryl, haloalkyl, haloalkoxy, -arylalkoxy, arylalkoxy-, cycloalkyl, —O-cycloalkyl, heterocyclyl, heteroaryl, alkylamino-, -alkyleneamine, alkoxyamino-, —O—CH₂-cycloalkyl; —COOR^{a}; —C(OR^{b}; —C(S) R^{a}; —C(O)NR^{a}R^{b}; —NR^{a}C(O)NR^{b}R^{c}; —N(R^{a})SOR^{b}; —N(R^{a}) SO₂R^{b}; —NR^{a}C(O)OR^{b}; —NR^{a}R^{b}; —NR^{a}C(O)R^{b}; —NR^{a}C(S)R^{b}; —SONR^{a}R^{b}; —SO₂NR^{a}R^{b}; —OR^{a}; —OR^{a}C(O)OR^{b}; —OC(O)NR^{a}R^{b}; —OC(O)R^{a}; —R^{a}NR^{b}R^{c}; —R^{a}OR^{b}; —SR^{a}; —SOR^{a} and —SO₂R^{a};
each of R^{a}, R^{b} and R^{c} are independently a hydrogen atom or optionally substituted alkyl, aryl, arylalkyl, cycloalkyl, heterocyclyl, heteroaryl or heteroarylalkyl;
   wherein the substituents are further optionally substituted by one or more halogen, hydroxy, nitro, cycloalkyl, cyano, azido, nitroso, amino, hydrazino, formyl, alkyl, alkoxy, oxo, aryl, alkoxyamino- or haloalkyl;
      wherein when the group R₁ is cyclic, R₁ is a residue formed by removing a hydrogen atom from a saturated or unsaturated monocyclic carbocyclic ring or a saturated or unsaturated monocyclic heterocyclic ring having one or two or three heteroatoms selected from a nitrogen atom, an oxygen atom and a sulfur atom in the ring, and a saturated or unsaturated monocyclic 3, 4, 5, 6, 7 or 8 membered heterocyclic ring having 3 to 5 carbon atoms, or a bicyclic or tricyclic condensed polycyclic heterocyclic ring having one or two or three heteroatoms in the ring, and a bicyclic or tricyclic condensed polycyclic heterocyclic ring having 7 to 13 carbon atoms, wherein the rings are optionally substituted with at least one substituent;
wherein R₂ is hydrogen or alkyl;
wherein R₃ is —NR^{d}R^{e};
   each of R^{a} and R^{e} are independently selected from the group consisting of hydrogen or an optionally substituted alkyl, alkenyl, aryl, arylalkyl, cycloalkyl, heterocyclyl, heteroaryl or heteroarylalkyl; or R^{d} and R^{e} together with the N, form an optionally substituted heterocyclic or heteroaryl ring;
      wherein the substituents are further optionally substituted by one or more halogen, hydroxy, nitro, cycloalkyl, cyano, azido, nitroso, amino, hydrazino, formyl, alkyl, aryl, heterocyclyl, alkoxy, mercapto, alkylthio, alkylcarbonyl, carboxy, alkxoycarbonyl, alkoxycarbonyloxy, oxo, or haloalkyl;
         wherein these further substituents are in turn further optionally substituted by one or more halogen, hydroxy, nitro, cycloalkyl, cyano, azido, nitroso, amino, hydrazino, formyl, alkyl, or haloalkyl;
their derivatives, analogs, tautomeric forms, stereoisomers, polymorphs, hydrates, solvates, intermediates, pharmaceutically acceptable salts, pharmaceutical compositions, metabolites and prodrugs thereof.

In a compound of formula I, R₁ may be hydrogen, optionally hydroxy-, amino-, carboxy-, C₁-C₆-alkyl-C₁-C₆ alkanoate-substituted C₁-C₂₀-alkyl, —C₂-C₂₀alkenyl, C₁-C₅-alkoxy-C₁-C₈-alkyl, or poly-C₁-C₈-alkoxy-C₁-C₈-alkyl, or represents mono-, bi-, or tricyclic C₃-C₁₃ cycloalkyl in which optionally one or two not directly adjacent methylene groups are replaced by oxygen and/or nitrogen, wherein cycloalkyl is optionally substituted with one or more halogen, hydroxyl, amino, oxo, phenyl or C₁-C₈-alkoxy, phenyl-C₁-C₆-alkyl, wherein phenyl is optionally substituted with halogen, hydroxyl, amino, C₁-C₆-alkyl, C₁-C₆-alkoxy, optionally halogen- or C₁-C₆-alkyl-substituted 5- or 6-membered heteroaryl having one or two heteroatoms selected from the group consisting of oxygen and nitrogen.

R₁ may be represented by any one of formula (IIa), (IIb), (IIc), (IId) or (IIe):

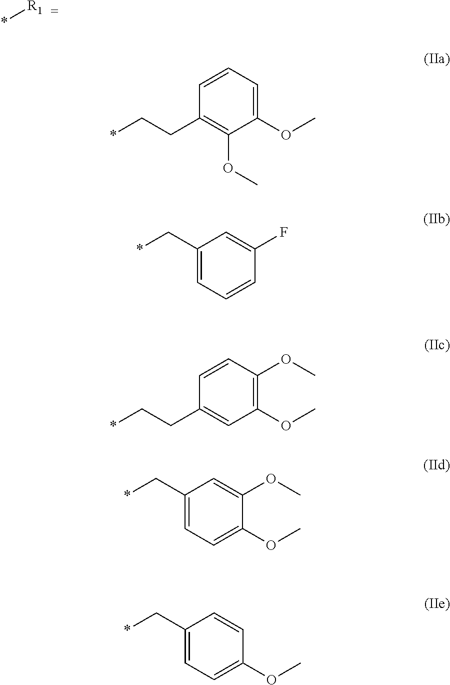

In a compound of formula I, R₂ may be hydrogen or C₁-C₆ alkyl.

In a compound of formula I, R₁R₂N may be selected from the groups consisting of:

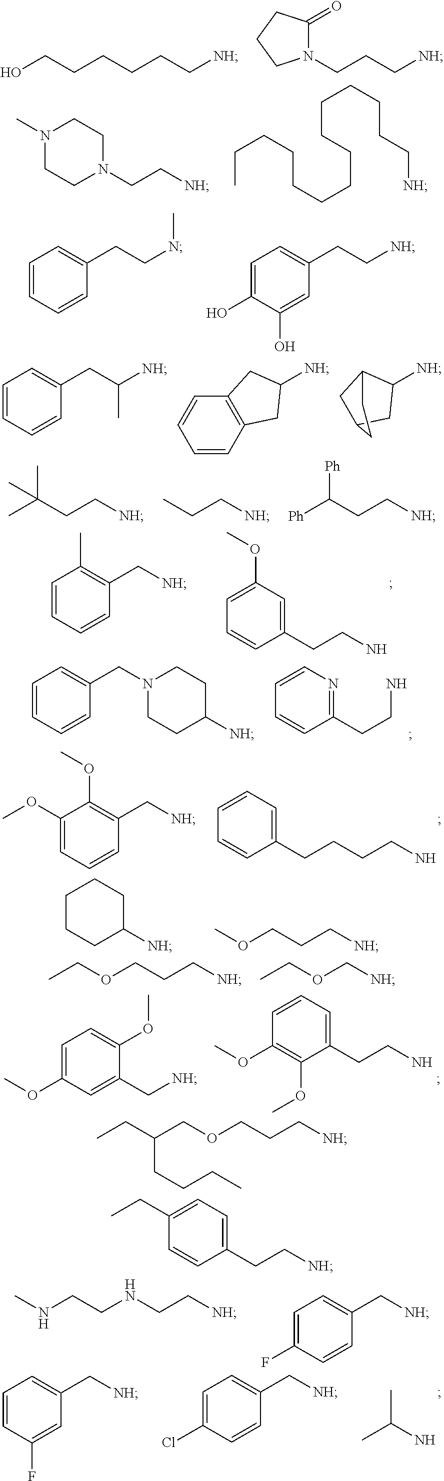

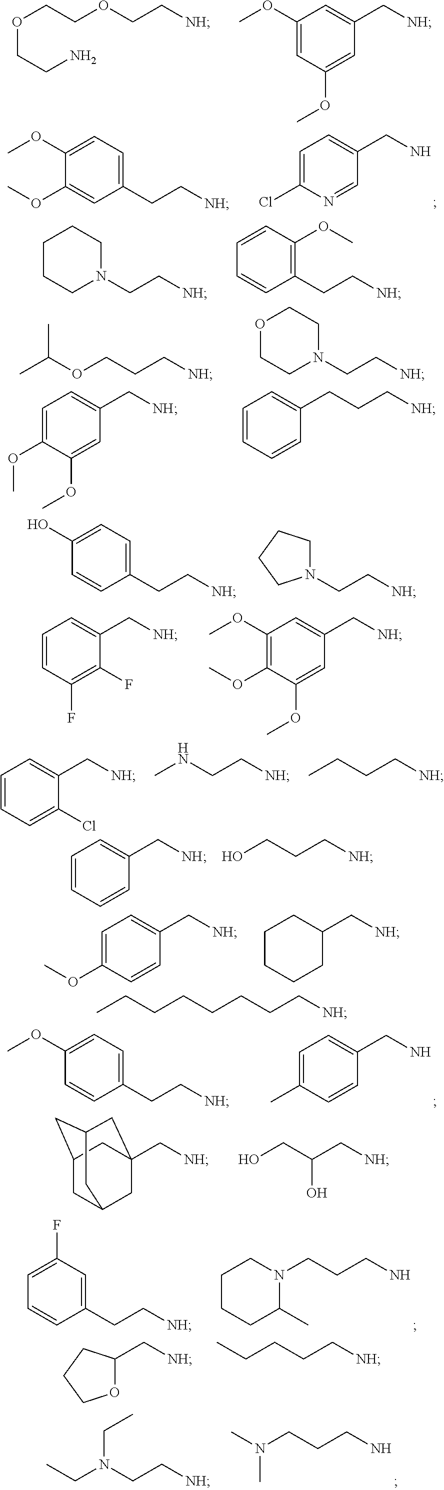

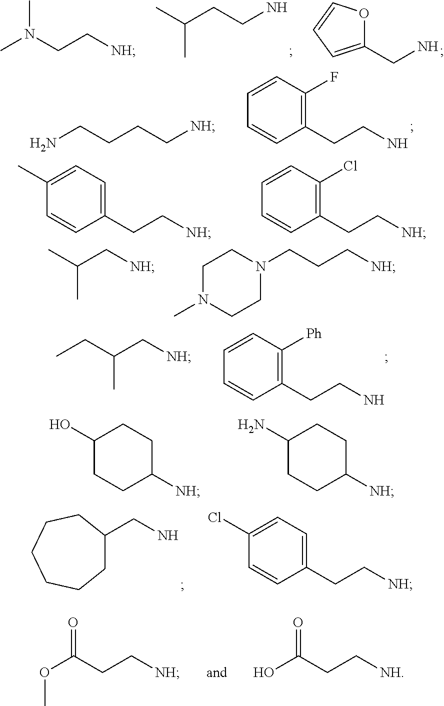

In a compound of formula I, R₃ may be represented by the formula —NR^{d}R^{e}, wherein each of R^{d} and R^{e} is independently selected from the group consisting of a hydrogen atom, or an optionally substituted group selected from the group consisting of:

an alkyl group having 1 to 20 carbon atoms which may have one or two or three substituents selected from the group consisting of:
a halogen atom, a cycloalkyl group having 3 to 10 carbon atoms, an aryl group, a residue formed by removing a hydrogen atom from a saturated or unsaturated monocyclic heterocyclic ring having one or two or three heteroatoms selected from the group consisting of a nitrogen atom, an oxygen atom and a sulfur atom in the ring, a saturated or unsaturated monocyclic 5 or 6 membered heterocyclic ring having 3 to 5 carbon atoms, a bicyclic or tricyclic condensed polycyclic heterocyclic ring having one or two or three heteroatoms in the ring, a bicyclic or tricyclic condensed polycyclic heterocyclic ring having 7 to 13 carbon atoms group, and an alkoxy group having an alkyl moiety having 1 to 8 carbon atoms;
   an alkenyl group having 2 to 8 carbon atoms;
   a cycloalkyl group having 3 to 10 carbon atoms;
   an aryl group having one or two or three substituents selected from the group consisting of:
a halogen atom, an alkyl group having 1 to 8 carbon atoms, a hydroxy group, an alkoxy group having an alkyl moiety having 1 to 8 carbon atoms, a mercapto group, an alkylthio group having an alkyl moiety having 1 to 8 carbon atoms, a formyl group, an alkylcarbonyl group having an alkyl moiety having 1 to 8 carbon atoms, a carboxy group, an alkoxycarbonyl group having an alkoxy group having an alkyl moiety having 1 to 8 carbon atoms, an alkylcarbonyloxy group having an alkylcarbonyl group having an alkyl moiety having 1 to 8 carbon atoms, a nitro group, a cyano group, a residue formed by removing a hydrogen atom from a saturated or unsaturated monocyclic heterocyclic ring having one or two or three heteroatoms selected from a nitrogen atom, an oxygen atom and a sulfur atom in the ring, and a saturated or unsaturated monocyclic 5 or 6 membered heterocyclic ring having 3 to 5 carbon atoms, or a bicyclic or tricyclic condensed polycyclic heterocyclic ring having one or two or three heteroatoms in the ring, and a bicyclic or tricyclic condensed polycyclic heterocyclic ring having 7 to 13 carbon atoms;
   the substituents which in turn are further optionally substituted by one or more halogen, hydroxy, nitro, cycloalkyl, cyano, azido, nitroso, amino, hydrazino, formyl, alkyl, haloalkyl.

R₃ may be selected from the group consisting of:

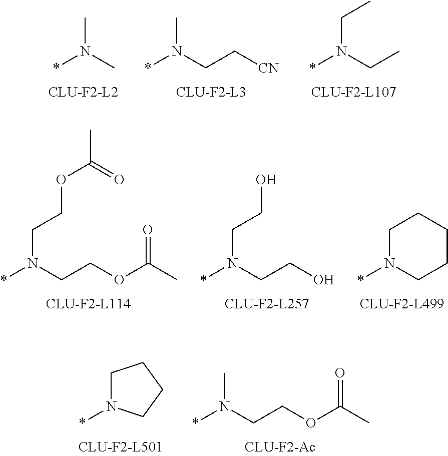

or their derivatives, analogs, tautomeric forms, stereoisomers, polymorphs, hydrates, solvates, intermediates, pharmaceutically acceptable salts, pharmaceutical compositions, metabolites and prodrugs thereof.

In a compound of Formula I, R^{d} and R^{e} may be represented independently by hydrogen, an optionally cyano-, hydroxy substituted C₁-C₂₀ alkyl, an optionally halogen-, hydroxy-, amino-, oxo-, C₁-C₆-alkoxy substituted C₃-C₁₀ cycloalkyl, wherein the cycloalkyl optionally has one or two heteroatoms from the group consisting of nitrogen and/or oxygen, or a C₁-C₆-alkyl-C₁-C₆-alkanoate.

The compound of formula I may be selected from the group consisting of:

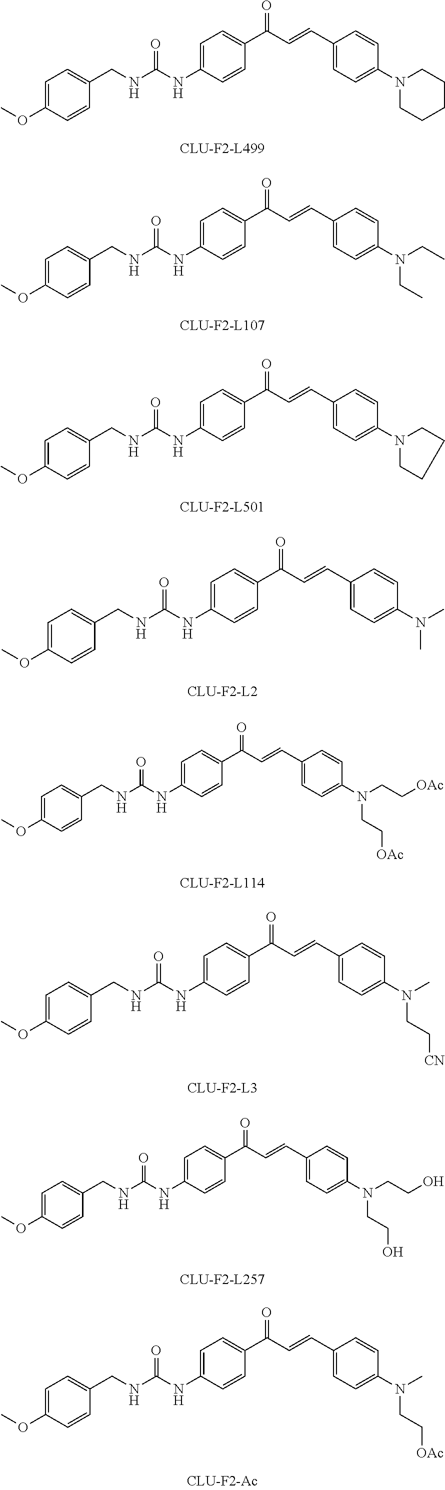

their derivatives, analogs, tautomeric forms, stereoisomers, polymorphs, hydrates, solvates, intermediates, pharmaceutically acceptable salts, pharmaceutical compositions, metabolites and prodrugs thereof.

As discussed above, the present disclosure provides a method of detecting insulin, wherein the method comprises the use of a compound described above.

Also as discussed above, the present disclosure provides a compound described above for use in detecting insulin.

Further as discussed above, the present disclosure provides the use of a compound described above for detecting insulin.

The insulin of the disclosed methods or uses may be detected in a sample selected from the group consisting of a cell culture, an isolated sample from a subject, a solution and an isolated cell from a tissue. The insulin may be selected from the group consisting of human, bovine, murine and porcine insulin. The insulin may comprise the amino acid sequence of SEQ ID NO. 1 to 8, wherein SEQ ID NO. 1 to 8 is the amino acid sequence of the A and B chains of human, bovine, porcine and murine insulin, respectively. See also Table 1.

The insulin may be encoded by a nucleic acid. The nucleic acid encoding the above-mentioned amino acid sequences would be encoded by codons as known in the art. For example, the nucleic acid may be selected from the group consisting of SEQ ID NOs: 9 to 16. See also Table 1a.

The nucleic acid may be comprised in a vector.

TABLE 1

| | SEQ | |
|---|---|---|
| Description | ID NO. | Amino Acid Sequence |
| Human insulin, | 1 | GIVEQCCTSICSLYQLENYCN |
| A chain | | |
| Human insulin, | 2 | FVNQHLCGSHLVEALYLVCGE |
| B chain | | RGFFYTPKT |
| Bovine insulin, | 3 | GIVEQCCASVCSLYQLENYCN |
| A chain | | |
| Bovine insulin, | 4 | PVNQHLCGSHLVEALYLVCGE |
| B chain | | RGFFYTPKA |
| Porcine insulin, | 5 | GIVEQCCTSICSLYQLENYCN |
| A chain | | |
| Porcine insulin, | 6 | FVNQHLCGSHLVEALYLVCGE |
| B chain | | RGFFYTPKA |
| Mouse insulin, | 7 | GIVDQCCTSICSLYQLENYCN |
| A chain | | |
| Mouse insulin, | 8 | FVKQHLCGPHLVEALYLVCGE |
| B chain | | RGFFYTPKS |

TABLE 1a

| | SEQ | |
|---|---|---|
| Description | ID NO. | Nucleic Acid Sequence |
| Human | 9 | ggcattgtggaacagtgctgcacca |
| insulin, A | | gcatttgcagcctgtatcagctgga |
| chain | | aaactattgcaac |
| Human | 10 | tttgtgaaccagcatctgtgcggca |
| insulin, B | | gccatctggtggaagcgctgtatct |
| chain | | ggtgtgcggcgaacgcggctttttt |
| | | tataccccgaaaacc |
| Bovine | 11 | ggcattgtggaacagtgctgcgcga |
| insulin, A | | gcgtgtgcagcctgtatcagctgga |
| chain | | aaactattgcaac |
| Bovine | 12 | tttgtgaaccagcatctgtgcggca |
| insulin, B | | gccatctggtggaagcgctgtatct |
| chain | | ggtgtgcggcgaacgcggctttttt |
| | | tataccccgaaagcg |
| Porcine | 13 | ggcattgtggaacagtgctgcacca |
| insulin, A | | gcatttgcagcctgtatcagctgga |
| chain | | aaactattgcaac |
| Porcine | 14 | tttgtgaaccagcatctgtgcggca |
| insulin, B | | gccatctggtggaagcgctgtatct |
| chain | | ggtgtgcggcgaacgcggctttttt |
| | | tataccccgaaagcg |
| Mouse | 15 | ggcattgtggatcagtgctgcacca |
| insulin, A | | gcatttgcagcctgtatcagctgga |
| chain | | aaactattgcaac |
| Mouse | 16 | tttgtgaaacagcatctgtgcggcc |
| insulin, B | | cgcatctggtggaagcgctgtatct |
| chain | | ggtgtgcggcgaacgcggctttttt |
| | | tataccccgaaaagc |

The detection of insulin in the disclosed methods or uses may comprise evaluating insulin secretion in a sample, wherein the sample is selected from the group consisting of a cell culture, an isolated sample from a subject, a solution and an isolated cell from a tissue.

The detection of insulin in the disclosed methods or uses may comprise the steps of:

a. contacting the test sample to be evaluated with a compound disclosed herein;

b. exciting the sample with a radiation source at a wavelength of about 430 nm, suitable to detect the compound;

c. observing the sample in conjunction with means for detecting fluorescence intensity of the compound at an emission wavelength of about 560 nm;

d. comparing the fluorescence in the test sample with the fluorescence in a control sample, wherein the control sample does not contain insulin;

wherein an increase in fluorescence intensity above a basal level of fluorescence in the control sample denotes the presence of insulin in the test sample.

The detection of insulin in the disclosed methods or uses may comprise evaluating secretion of insulin by living cells, wherein said step of contacting the sample with a compound disclosed herein comprises incubating said cells in a medium containing said compound for sufficient time, such that said insulin is secreted in the medium where said insulin can be detected.

The evaluation of secretion of insulin may comprise means for detecting fluorescence of a disclosed compound in real time, thereby providing real time monitoring of secretion of insulin by living cells.

The compound of the disclosed methods or uses may be:

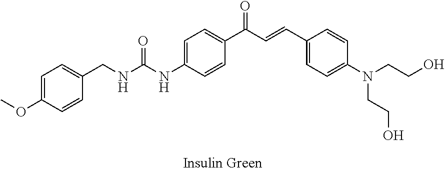 [SMILES: COc1ccc(CNC(=O)Nc2ccc(C(=O)/C=C/c3ccc(N(CCO)CCO)cc3)cc2)cc1]

As discussed above, there is provided a method for screening a compound that is suspected to affect secretion of insulin comprised in a test sample, comprising the steps of:

a. contacting the compound to be screened with the test sample;

b. contacting a compound disclosed herein with the test sample, wherein contacting is simultaneous, sequential or separate;

c. detecting a signal from the compound as disclosed herein, which is indicative of the presence of insulin in the test sample to obtain a first value; and

d. comparing the first value with a control value, wherein the control value corresponds to a signal in a control sample, and the control value is determined from the control sample which is the same as provided in (a), except that the control sample is not contacted with the compound to be screened;

wherein if the test value is greater or smaller than the control value, then the compound affects secretion of insulin in the sample.

In the disclosed method, the test sample may be selected from the group consisting of a cell culture, an isolated sample from a subject and an isolated islet.

In the disclosed method, the contacting of step b may be done simultaneously, separately or sequentially.

In the disclosed method, the detecting of step c may be real time detection.

In the disclosed method, the compound to be screened may be selected from the group consisting of KCl, glucose, Forskolin ((3R,4aR,5S,6S,6aS,10S,10aR,10bS)-6,10,10b-trihydroxy-3,4a,7,7,10a-pentamethyl-1-oxo-3-vinyldodecahydro-1H-benzo[f]chromen-5-yl acetate), 3-isobutyl-1-methylxanthine (IBMX; 1-methyl-3-(2-methylpropyl)-7H-purine-2,6-dione), Glibenclamide (5-chloro-N-(4-[N-(cyclohexylcarbamoyl)sulfamoyl]phenethyl)-2-methoxybenzamide), amiodarone ((2-{4-[(2-butyl-1-benzofuran-3-yl)carbonyl]-2,6-diiodophenoxy}ethyl)diethylamine), nifedipine (3,5-dimethyl 2,6-dimethyl-4-(2-nitrophenyl)-1,4-dihydropyridine-3, 5-dicarboxylate), Leucine, Tolbutamide (N-[(butylamino)carbonyl]-4-methylbenzenesulfonamide), Ouabain (1β,3β,5β,11α,14,19-Hexahydroxycard-20 (22)-enolide 3-(6-deoxy-a-L-mannopyranoside), 4-[(1R,3S,5S,8R,9S,10R,11R,13R,14S,17R)-1,5,11,14-tetrahydroxy-10-(hydroxymethyl)-13-methyl-3-((2R,3R,4R,5R,6S)-3,4,5-trihydroxy-6-methyltetrahydro-2H-pyran-2-yloxy)hexadecahydro-1H-cyclopenta[a]phenanthren-17-yl]furan-2(5H)-one) and adrenalin.

As discussed above, there is provided a method of assessing whether a condition or a stimulus affect secretion of insulin in cultured cells, comprising:
a. culturing cells expressing insulin under a condition or stimulus to be assessed for its effect on secretion of insulin, wherein the cells are cultured in the presence of a compound as disclosed herein, wherein the cells are referred to as test cells;
b. detecting a signal from the compound, which is indicative of the presence of insulin in an extracellular environment of the test cells to obtain a first value;
c. comparing the first value with a control value, wherein the control value corresponds to a signal in the extracellular environment of the control cells, and the control value is determined from control cells which are the same cells as cultured in (a), and which are cultured under the same conditions as in (a), except that the control cells are not cultured under the condition or stimulus to be assessed,
wherein if the test value is smaller or greater than the control value, then the condition or stimulus affects secretion of insulin in the cells.

In the disclosed method, the condition or stimulus to be assessed may be selected from the group consisting of one or more pH, temperature, pressure, medium composition comprising serum, salts, vitamins, hormones, proteins, carbohydrates, lipids and chemical compounds, age and origin of the culture, atmosphere composition comprising CO₂ and O₂ concentration, cell culture support comprising two-dimensional culture, three-dimensional scaffold, and suspension culture.

In the disclosed method, the detecting of step c may be real time detection.

In the disclosed method, the compound may be:

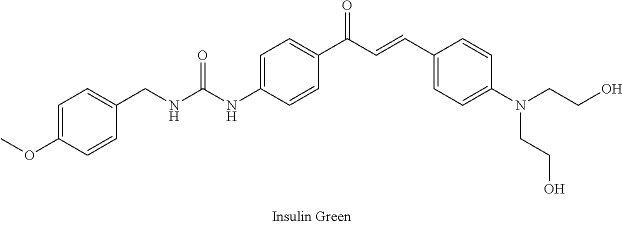 [SMILES: COc1ccc(CNC(=O)Nc2ccc(C(=O)/C=C/c3ccc(N(CCO)CCO)cc3)cc2)cc1]

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1a is a graph showing the fluorescence response of Insulin Green to different concentrations of insulin.

FIG. 1b is a graph showing the interactions between Insulin Green and insulin.

FIG. 1c is a graph showing the selectivity of Insulin Green to insulin over bovine serum albumin (BSA), human serum albumin (HSA) and HSA lipid free.

FIG. 2 is a graph showing the quantification of insulin concentration released from cultured beta cells.

FIG. 3a is a graph showing the fluorescence response of Insulin Green with cultured beta cells challenged with four different conditions: Series 1: Glucose concentration 2.8 mM was used as control; Series 2: 16.7 mM glucose and 1 μM of forskoline to induce insulin release; Series 3: Insulin release was further stimulated with 16.7 mM glucose, 5 μM forskoline and 100 μM IBMX; Series 4: Adrenaline was pre-incubated with the cells for 5 min to inhibit insulin secretion upon the treatment of series 3.

FIG. 3b is a graph showing the fluorescence response of Insulin Green with isolated islets challenged with four different conditions: Series 1: Glucose concentration 2.8 mM was used as control; Series 2: 16.7 mM glucose and 1 μM of forskoline to induce insulin release; Series 3: Insulin release was further stimulated with 16.7 mM glucose, 5 μM forskoline and 100 μM IBMX; Series 4: Adrenaline was pre-incubated with the cells for 5 min to inhibit insulin secretion upon the treatment of series 3.

FIG. 4 shows the structure of Formula (I).

### EXAMPLES

Non-limiting examples of the invention will be further described in greater detail by reference to specific Examples, which should not be construed as in any way limiting the scope of the invention.

### 1. Materials and Methods

All the reagents and solvents were purchased from Aldrich, Alfa and Acros organics and used without further purification. All reactions were performed in oven-dried glassware under a positive pressure of nitrogen. Analytical TLC was carried out on Merck 60 F254 silica gel plate (0.25 mm layer thickness) and visualization was done with UV light. Column chromatography was performed on Merck 60 silica gel (230-400 mesh). NMR spectra were recorded on a Bruker Avance 300 NMR spectrometer. Chemical shifts are reported as 6 in units of parts per million (ppm) and coupling constants are reported as a J value in Hertz (Hz). Mass of all the compounds was determined by LC-MS of Agilent Technologies with an electrospray ionization source. Spectroscopic measurements were performed on a fluorometer and UV/VIS instrument, Synergy 4 of bioteck company and Gemini XS fluorescence plate reader. The slit width was 1 nm for both excitation and emission.

1a. Cell Culture

Beta TC6 cell was cultured in high-glucose (4500 mg/L) contained Dulbecco's Modified Eagle's medium (DMEM) supplemented with 10% fetal bovine serum, 100 U/mL penicillin, 100 μg/ml streptomycin, and incubated at 37° C. with 5% CO2. Cells were plated on plates 24-36 hours prior to experiment.

1b. Isolation of Islets

Extracted pancreas is immediately transferred into a vial containing cold Krebs-Ringer-Hepes (KRH) medium (containing, mM: 130 NaCl, 4.7 KCl, 1.2 KH₂PO₄, 1.2 MgSO₄, and 2.56 CaCl₂, 1 mg/mL BSA, 20 mM HEPES, pH 7.4) and cut into smaller pieces. 0.5 mg/mL of Collagenase-P (Roche-applied-science) was added and placed into a 37° C. water bath shaker (180-200 rpm/min) for 15-20 min. After digestion, cold KRH buffer with 0.1% BSA was added and inverted several times. The suspension was washed away after allowing islets to settle to the bottom of the vial for 10 mins and this wash process was repeated twice more.

### Example 1: Calcone-Urea (CLU) Library

The present invention includes the synthesis of fluorescent chalcone-urea library (CLU), which was prepared on solid support. The CLU library was screened against four different concentrations of insulin (0.2, 0.1, 0.05 and 0.025 mg/mL) in HEPES buffer (20 mM, pH=7.4). Based on fluorescent intensity, 5 hits with more than 50-fold increase were selected (Table 2). CLU-381 was further derivatized due to the synthetic convenience to render 8 derivatives (Table 3). These 8 compounds were then tested against insulin to identify CLU-381-L257 (Insulin Green) as the most responsive compound.

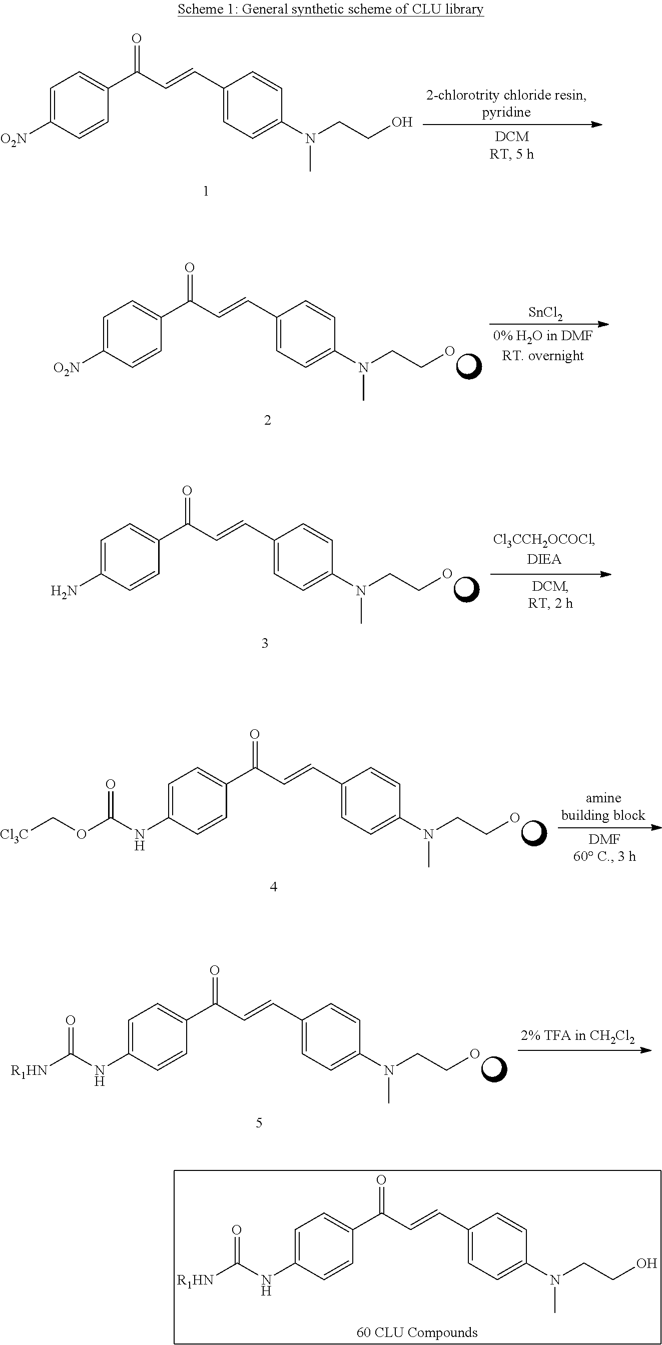

TABLE 2

| CLU | |
|---|---|
| compound | R₁ |
| CLU-C5 | 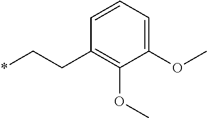 [SMILES: *CCc1cccc(OC)c1OC] |
| CLU-C10 | 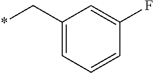 [SMILES: *Cc1cccc(F)c1] |
| CLU-D5 | 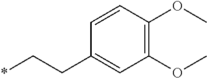 [SMILES: *CCc1ccc(OC)c(OC)c1] |
| CLU-D11 | 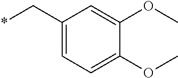 [SMILES: *Cc1ccc(OC)c(OC)c1] |
| CLU-F2 | 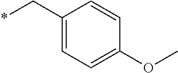 [SMILES: *Cc1ccc(OC)cc1] |

### 1.1: Synthesis of Compound 1

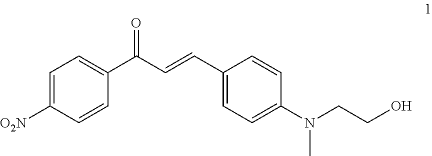 [SMILES: CN(CCO)c1ccc(/C=C/C(=O)c2ccc([N+](=O)[O-])cc2)cc1]

To a solution of 4′-Nitroacetophenone (100 mg, 0.6 mmole) and N,N′2-4-((2-hydroxyethyl)(methyl)amino)benzaldehyde (100 mg, 0.55 mmole) in EtOH, pyrrolidine 0.1 mL was added and heat up with commercial Microwave Reactor (150 MW) for 3 min. Resulted. solution was cooled down and kept at the room temperature for 2 hrs to precipitate dark red solid. Solid was filtered and washed with ethylacetate and hexane (1:1) solution and dried as a dark red solid (100 mg, 60%). Obtained solid was used for further reaction without any purification. 1H-NMR (DMSO-d6) δ 8.34 (m, 4H), 7.71 (m, 4H), 6.75 (d, J=9, 2H), 4.75 (bt, 1H), 3.57 (bt, 2H), 3.49 (bt, 2H), 3.03 (s, 3H); 13C-NMR (CDCl₃) δ 187.4, 151.6, 149.4, 146.9, 143.5, 131.4, 129.5, 128.3, 125.4, 123.7, 121.3, 115.3, 111.5, 58.2, 53.9, ESI-MS m/z (M+H) calc'd: 323.1, found 323.0.

### 1.2: Synthesis of Compound 2

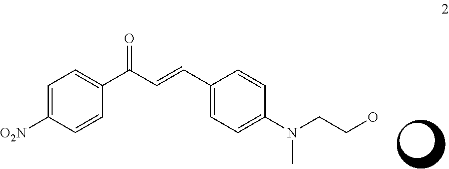 [SMILES: CN(CCO)c1ccc(/C=C/C(=O)c2ccc([N+](=O)[O-])cc2)cc1]

2-ChloroTrityl Resin (200 mg, 1 mmole/g) was pre-swelled in DCM. To the Resin solution compound 1 (100 mg) and 3 eq. of pyridine were added and kept for overnight. The reaction mixture was filtered and washed with DMF, MeOH, DCM after 1 hr MeOH capping procedure for extra resin chloride deactivation. Washed resin was dried with high vacuum dessicator and used for further reaction.

### 1.3: Synthesis of Compound 5

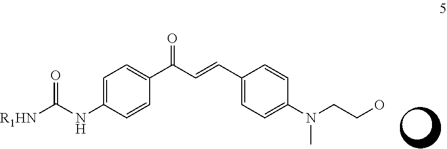

Resin loaded compound 2 was treated with SnCl₂ hydrate eq. in DMF solution. Reaction mixture was shaken overnight and washed with DMF, MeOH, DCM. DCM swelled resin was placed in 50 mL tube and trichloroehtyl chlorocarbonate (3 eq) was added with DIEA (3 eq) at room temperature. After shaking for 2 hours the reaction mixture was washed with DMF, MeOH, DCM.

After high vacuum drying, the resin was divided by 60 mg each in the 5 mL syringe. The reaction syringes were washed with DMF and amines (3 eq) in DMF 3 mL was loaded in syringe and shake at 60° C. for 3 hrs. After reaction the reaction syringes were washed out with DCM, MeOH, DCM 3 times each.

### 1.4: Cleavage Protocol

2% TFA in DCM solution was loaded for cleavage. For 30 min, incubated with cleavage solution, aliquot was squeezed out and collected in 20 mL vial. 2.5% ammonia water in ACN was added for TFA neutralization and filter with silica end filled Tip. Dark brown solid was obtained after removing solvent and purity was analyzed by LC/MS instrument. LC/MS gradient condition was 5% ACN to 100% ACN in water with 4.3×50 mm C18 column. All HPLC solvent contains 0.1% formic acid for the LC/MS analysis.

### Example 2: CLU-381 Derivatives

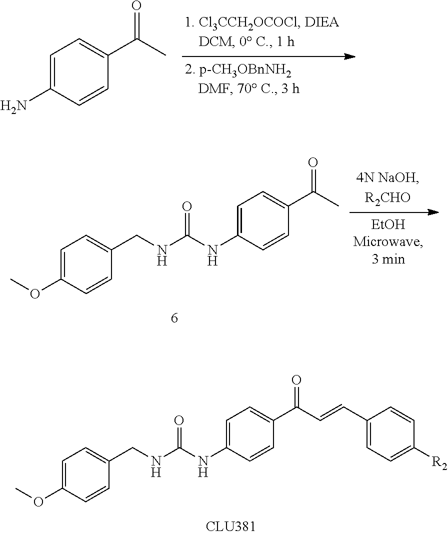

### Scheme 2: General Synthetic Scheme for CLU-381 Derivatives

To an EtOH soln. (40 ml) of the acetophenone (1 mmol each) and a mixture of the aldehyde (1.5 mmol) was added a solution of aq. 4 N NaOH (0.5 mL). The mixture was exposed to microwave irradiation until EtOH is dried. The mass obtained was cooled and neutralized with cold dil. aq. HCl. The chalcones were successively extracted with AcOEt (50 mL) and purified by column chromatography in the solvent system of MeOH (0 to 5%) in DCM.

### 2.1: Synthesis of Compound 6

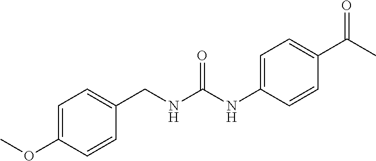 [SMILES: COc1ccc(CNC(=O)Nc2ccc(C(C)=O)cc2)cc1]

In the 50 mL of round bottomed flask, 4′-aminoacetophenon was dissolved in DCM with DIEA, at 0° C. the trichloroethoxychloroformate was added slowly. After reaction completion reaction mixture was concentrated and redissolved with DMF, p-methoxybenzyl amine (3 eq) addition was followed and heat to 70° C. for 3 hrs. Reaction mixture was concentrated with vacuum and purified with column chromatography on silica. Reaction yield was 90%. 1H-NMR (CDCl₃+CD3OD) δ 7.80 (d, J=8.4, 2H), 7.42 (d, J=8.4, 2H), 7.18 (d, J=8.1, 2H), 6.80 d, J=8.1, 2H), 4.29 (s, 2H), 3.73 (s, 3H), 2.49 (s, 3H); 13C-NMR (CDCl3+CD3OD) δ 187.4, 151.6, 149.4, 146.9, 143.5, 131.4, 129.5, 128.3, 125.4, 123.7, 121.3, 115.3, 111.5, 58.2, 53.9.

TABLE 3

| CLU381 | | Compound |
|---|---|---|
| derivative | R₂ | structure |
| CLU-F2-L2 | 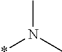 [SMILES: *N(C)C] | 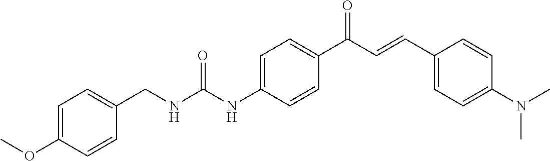 |
| CLU-F2-L3 | 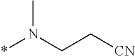 [SMILES: *N(C)CCC#N] | 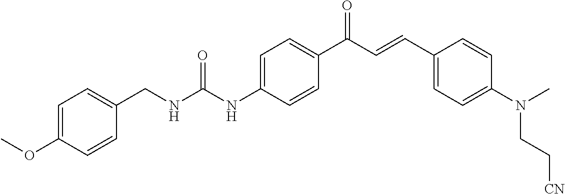 [SMILES: COc1ccc(CNC(=O)Nc2ccc(C(=O)/C=C/c3ccc(N(C)CCC#N)cc3)cc2)cc1] |
| CLU-F2-L107 | 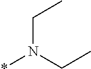 [SMILES: *N(CC)CC] | 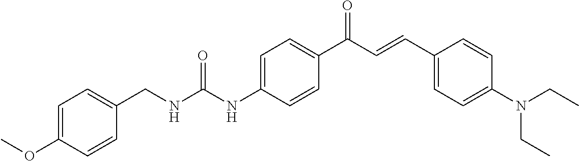 [SMILES: CCN(CC)c1ccc(/C=C/C(=O)c2ccc(NC(=O)NCc3ccc(OC)cc3)cc2)cc1] |
| CLU-F2-L114 | 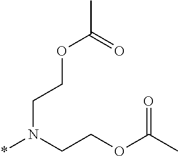 [SMILES: *N(CCOC(C)=O)CCOC(C)=O] | 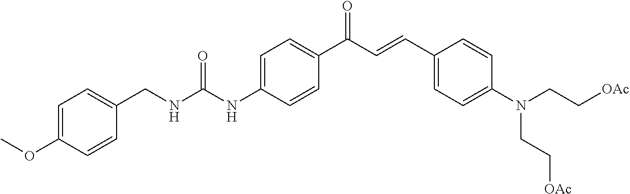 [SMILES: COc1ccc(CNC(=O)Nc2ccc(C(=O)/C=C/c3ccc(N(CCOC(C)=O)CCOC(C)=O)cc3)cc2)cc1] |
| CLU-F2-L257 | 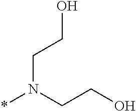 [SMILES: *N(CCO)CCO] | 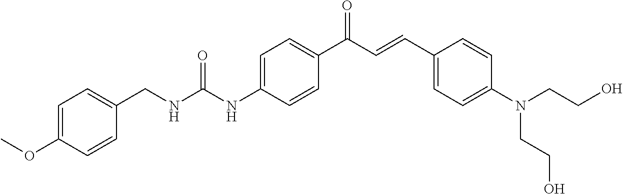 [SMILES: COc1ccc(CNC(=O)Nc2ccc(C(=O)/C=C/c3ccc(N(CCO)CCO)cc3)cc2)cc1] |
| CLU-F2-L499 | 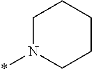 [SMILES: *N1CCCCC1] | 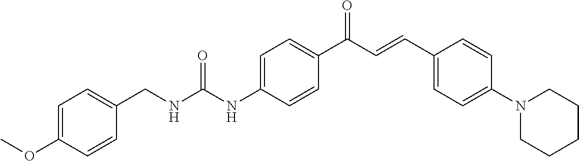 [SMILES: COc1ccc(CNC(=O)Nc2ccc(C(=O)/C=C/c3ccc(N4CCCCC4)cc3)cc2)cc1] |
| CLU-F2-L501 | 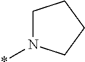 [SMILES: *N1CCCC1] | 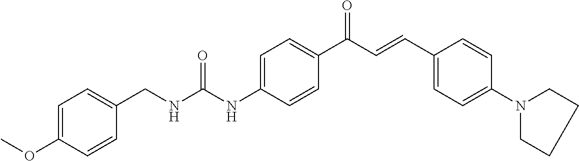 [SMILES: COc1ccc(CNC(=O)Nc2ccc(C(=O)/C=C/c3ccc(N4CCCC4)cc3)cc2)cc1] |
| CLU-F2-Ac | 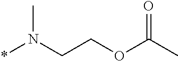 [SMILES: *N(C)CCOC(C)=O] | 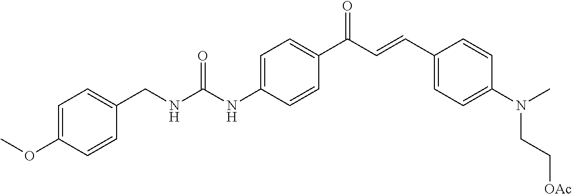 [SMILES: COc1ccc(CNC(=O)Nc2ccc(C(=O)/C=C/c3ccc(N(C)CCOC(C)=O)cc3)cc2)cc1] |

TABLE 4

| CLU381 | Compound |
|---|---|
| derivative | structure |
| CLU-F2-L2 | 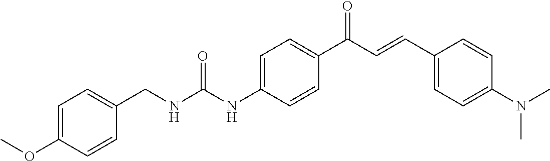 |
| CLU-F2-L3 | 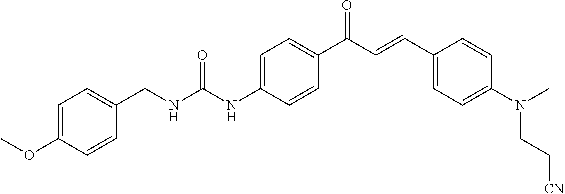 [SMILES: COc1ccc(CNC(=O)Nc2ccc(C(=O)/C=C/c3ccc(N(C)CCC#N)cc3)cc2)cc1] |
| CLU-F2-L107 | 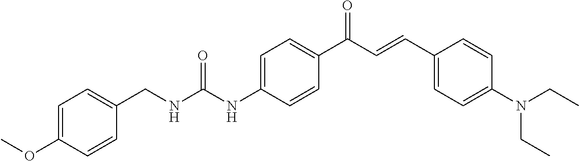 [SMILES: CCN(CC)c1ccc(/C=C/C(=O)c2ccc(NC(=O)NCc3ccc(OC)cc3)cc2)cc1] |
| CLU-F2-L114 | 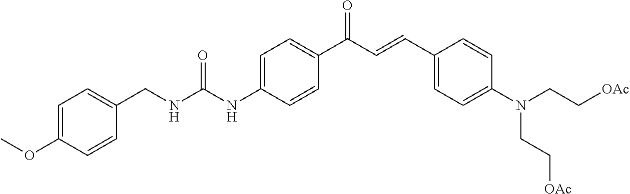 [SMILES: COc1ccc(CNC(=O)Nc2ccc(C(=O)/C=C/c3ccc(N(CCOC(C)=O)CCOC(C)=O)cc3)cc2)cc1] |
| CLU-F2-L257 | 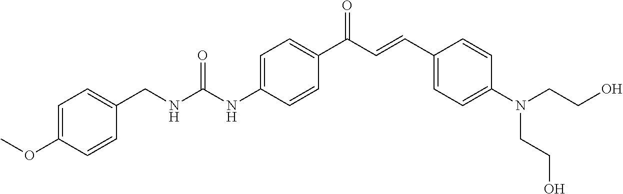 [SMILES: COc1ccc(CNC(=O)Nc2ccc(C(=O)/C=C/c3ccc(N(CCO)CCO)cc3)cc2)cc1] |
| CLU-F2-L499 | 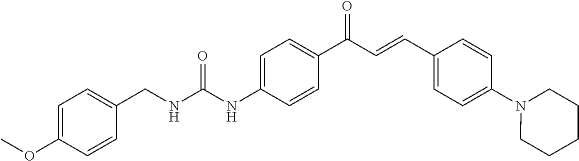 [SMILES: COc1ccc(CNC(=O)Nc2ccc(C(=O)/C=C/c3ccc(N4CCCCC4)cc3)cc2)cc1] |
| CLU-F2-L501 | 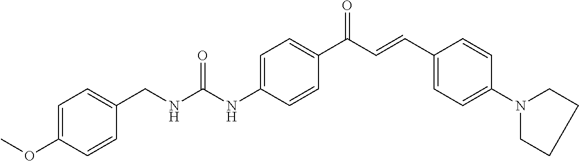 [SMILES: COc1ccc(CNC(=O)Nc2ccc(C(=O)/C=C/c3ccc(N4CCCC4)cc3)cc2)cc1] |
| CLU-F2-Ac | 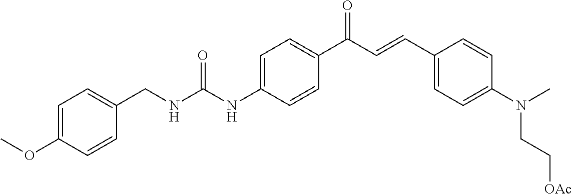 [SMILES: COc1ccc(CNC(=O)Nc2ccc(C(=O)/C=C/c3ccc(N(C)CCOC(C)=O)cc3)cc2)cc1] |

### Example 3: R₁-Amine Building Blocks Used in the Synthesis of CLU Library

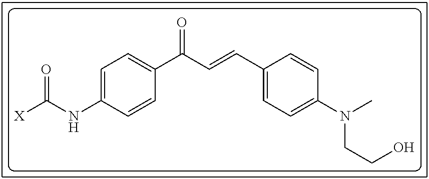

| Code | Structure |
|---|---|
| Amine-6 | 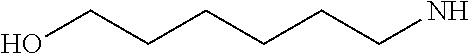 |
| Amine-11 | 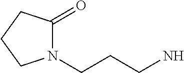 |
| Amine-25 | 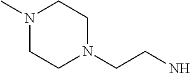 |
| Amine-28 | 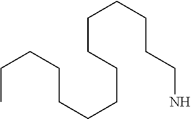 |
| Amine-33 | 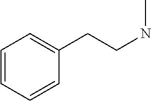 |
| Amine-48 | 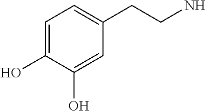 [SMILES: NCCc1ccc(O)c(O)c1] |
| Amine-77 | 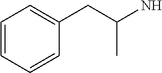 |
| Amine-78 | 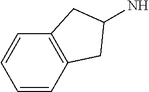 |
| Amine-89 | 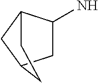 |
| Amine-92 | 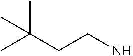 |
| Amine-95 | 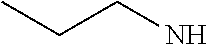 |
| Amine-100 | 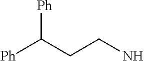 [SMILES: NCCC(c1ccccc1)c1ccccc1] |
| Amine-101 | 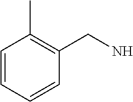 |
| Amine-102 | 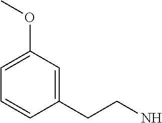 |
| Amine-107 | 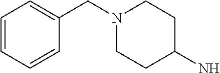 [SMILES: NC1CCN(Cc2ccccc2)CC1] |
| Amine-111 | 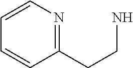 |
| Amine-124 | 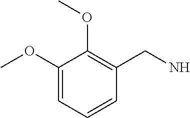 |
| Amine-131 | 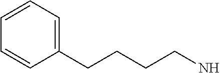 [SMILES: NCCCCc1ccccc1] |
| Amine-164 | 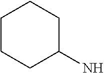 |
| Amine-165 | 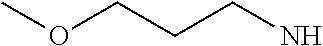 |
| Amine-167 | 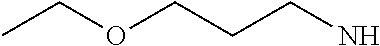 |
| Amine-177 | 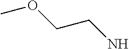 |
| Amine-181 | 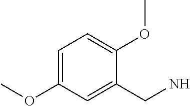 |
| Amine-184 | 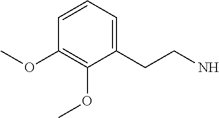 |
| Amine-193 | 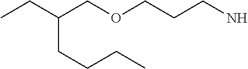 |
| Amine-201 | 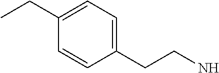 |
| Amine-216 | 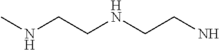 |
| Amine-218 | 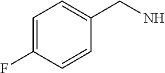 |
| Amine-220 | 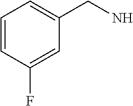 |
| Amine-221 | 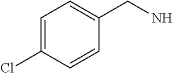 |
| Amine-222 | 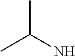 |
| Amine-225 | 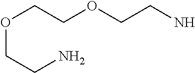 |
| Amine-262 | 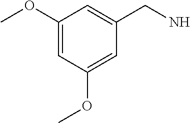 |
| Amine-266 | 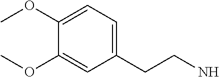 |
| Amine-267 | 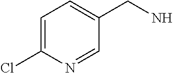 |
| Amine-272 | 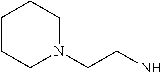 |
| Amine-273 | 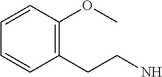 |
| Amine-274 | 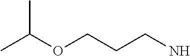 |
| Amine-282 | 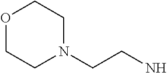 |
| Amine-319 | 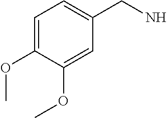 |
| Amine-335 | 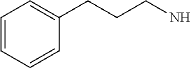 |
| Amine-346 | 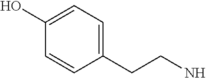 |
| Amine-347 | 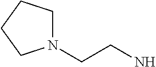 |
| Amine-351 | 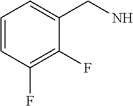 |
| Amine-356 | 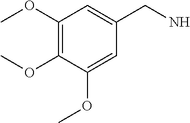 |
| Amine-364 | 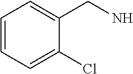 |
| Amine-370 | 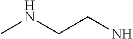 |
| Amine-373 | 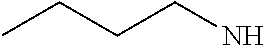 |
| Amine-374 | 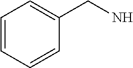 |
| Amine-376 | 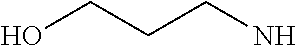 |
| Amine-381 | 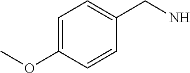 |
| Amine-382 | 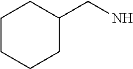 |
| Amine-384 | 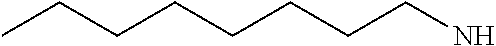 |
| Amine-395 | 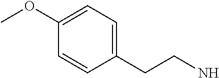 |
| Amine-396 | 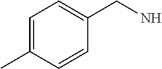 |
| Amine-397 | 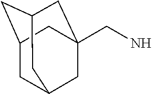 [SMILES: NCC12CC3CC(CC(C3)C1)C2] |
| Amine-404 | 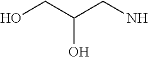 |
| Amine-405 | 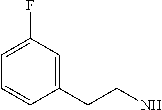 |
| Amine-414 | 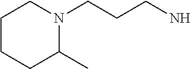 |
| Amine-417 | 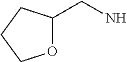 |
| Amine-419 | 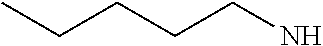 |
| Amine-420 | 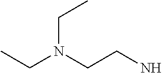 |
| Amine-427 | 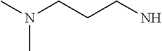 |
| Amine-434 | 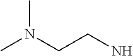 |
| Amine-442 | 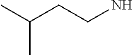 |
| Amine-446 | 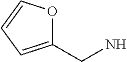 |
| Amine-454 | 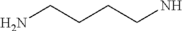 |
| Amine-477 | 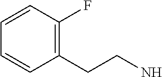 |
| Amine-478 | 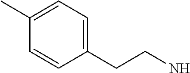 |
| Amine-480 | 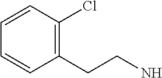 |
| Amine-487 | 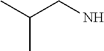 |
| Amine-526 | 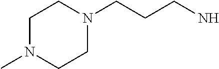 |
| Amine-574 | 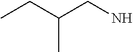 |
| Amine-599 | 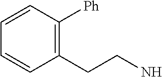 [SMILES: NCCc1ccccc1-c1ccccc1] |
| Amine-615 | 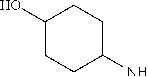 |
| Amine-616 | 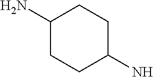 |
| Amine-618 | 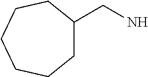 |
| Amine-677 | 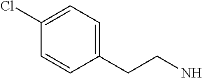 |
| Amine-N154 | 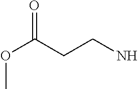 |
| Amine-N154A | 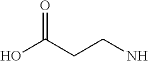 |

### Example 4: Insulin Green

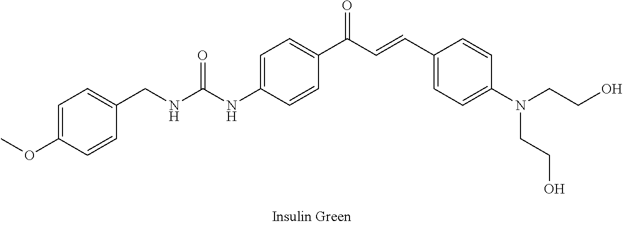 [SMILES: COc1ccc(CNC(=O)Nc2ccc(C(=O)/C=C/c3ccc(N(CCO)CCO)cc3)cc2)cc1]

1H-NMR (CDCl3+CD3OD) δ 7.84 (d, J=8.7, 2H), 7.63 (d, J=15.4, 1H), 7.42 (m, 4H), 7.25 (d, J=15.4, 1H), 7.14 (d, J=8.7, 2H), 6.76 (d, J=8.7, 2H), 6.62 (d, J=8.8, 2H), 4.25 (s, 2H), 3.72 (t, J=5.2, 4H), 3.68 (s, 3H), 3.52 (t, J=5.1, 4H), 13C-NMR (CDCl₃+CD₃OD) δ 189.8, 149.8, 145.2, 144.0, 130.3, 129.6, 128.5, 117.2, 113.7, 111.8, 59.6, 55.0, 54.5, 42.9, 29.4.

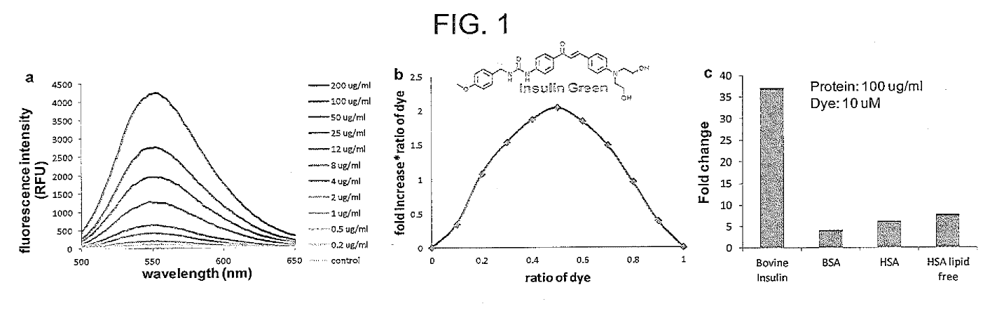

Insulin Green showed a 94-fold fluorescent increase to 200 μg/mL insulin (FIG. 1a). The job plot reveals that a maximum fluorescence increase was observed for 1:1 insulin: Insulin Green ratio (FIG. 1b). Furthermore, Insulin Green also showed good selectivity over other proteins, such as the most abundant protein in blood (i.e., serum albumin) (FIG. 1c).

The good sensitivity and selectivity of Insulin Green displays its potential in a wide variety of applications.

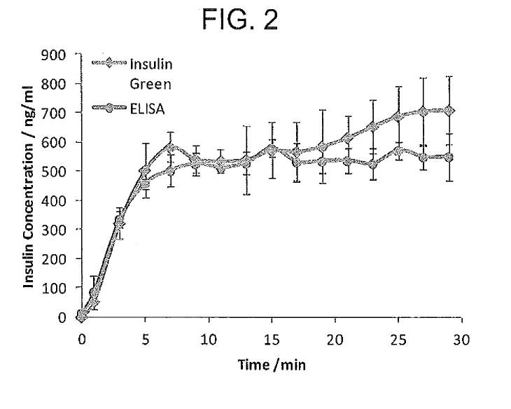

Insulin quantification in cell culture medium was explored using Insulin Green. KCl is known to induce the insulin release from both beta cell and isolated islet. After beta cell was plated on 6-well plates, the cells were pre-incubated for 30 min in 2.8 mM glucose and then challenged with 50 mM KCl. The media was collected at 2 min intervals and measured with both Insulin Green and enzyme-linked immunosorbent assay (ELISA). The results correlate well with each other, indicating that Insulin Green can provide a fast way for insulin quantification (FIG. 2). However, the real time dynamics are still beyond observation due to time averaging.

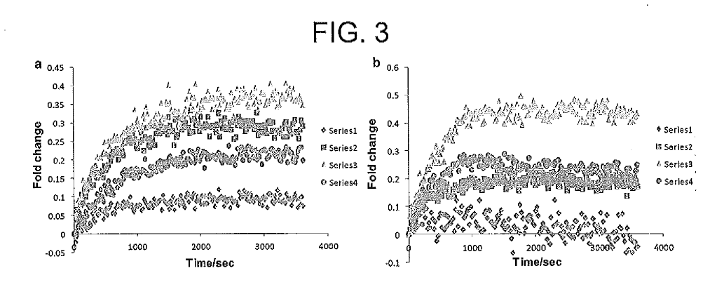

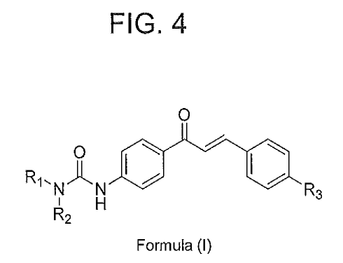

Therefore, Insulin Green was applied to the real-time measurement of insulin secretion from both cultured cells and isolated islets. Forskolin and 3-isobutyl-1-methylxanthine (IBMX) are known to be able to stimulate insulin release, while adrenaline can inhibits insulin secretion. After beta cells were plated on 96-well plates, cell were pre-incubated for 30 min in 2.8 mM glucose and treated with 10 μM Insulin Green. To test whether Insulin Green can respond the dynamic insulin concentration change in real time, the cells were further challenged with four different conditions. With reference to FIGS. 3a and 3b, the glucose concentration remaining as 2.8 mM was used as control (series 1). The cells were challenged with 16.7 mM glucose and 1 μM of forskoline to induce insulin release (series 2). The insulin release was further stimulated with 16.7 mM glucose, 5 μM forskoline and 100 μM IBMX (series 3). Adrenaline was pre-incubated to the cells for min to inhibit insulin secretion upon the treatment of series 3 (series 4). After the cells were challenged with four different conditions, the 96-well plates were immediately applied to a fluorescent plate reader to monitor the fluorescence change of Insulin Green at 20 seconds intervals. The fluorescence changes of Insulin Green showed clear differences with different conditions and correlated well with reported values. Similar experiments were also performed on isolated islets. Insulin Green showed similar response to the four different conditions. These results indicate that Insulin Green can measure the insulin secretion from both cultured cells and isolated islets in real time.

### Applications

The disclosed compounds may be useful as insulin sensors.

The disclosed compounds may display good sensitivity and selectivity for insulin.

The disclosed compounds may be able to detect insulin rapidly and directly.

The disclosed compounds may display sensitive fluorescent intensity enhancement to insulin.

The disclosed methods may allow for fast detection of insulin and may display low detection limits.

The disclosed methods may provide real time monitoring of insulin secretion in both cultured cells and isolated islets.

The disclosed methods may not require labelling insulin with expensive antibodies, fluorescent tags or radioisotopes. Therefore, the disclosed methods may be cost-effective.

The disclosed methods may be simple to perform and may not require time consuming preparation steps.

The disclosed methods may provide for fast insulin detection and insulin secretion study in both cultured cells and isolated islets.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A compound of formula (I) 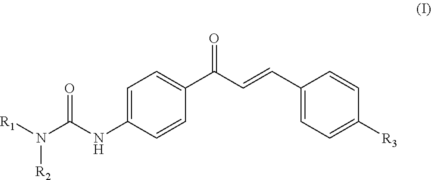
wherein the configuration around the double bond is E or Z;
wherein R₁ is selected from the group consisting of:
hydrogen,
cyclic aliphatic, acyclic aliphatic, branched aliphatic, unbranched aliphatic, substituted aliphatic, unsubstituted aliphatic,
cyclic heteroaliphatic, acyclic heteroaliphatic, branched heteroaliphatic, unbranched heteroaliphatic, substituted heteroaliphatic, unsubstituted heteroaliphatic,
substituted aryl, unsubstituted aryl,
substituted heteroaryl, unsubstituted heteroaryl,
cyclic acyl, acyclic acyl, substituted acyl and unsubstituted acyl;
wherein when the group R₁ is substituted, the one or more substituents are selected from the group consisting of halogen, hydroxyl, nitro, cyano, oxo (═O), thioxo (═S), azido, nitroso, amino, hydrazine, formyl, alkyl, alkene, alkyne, -alkyalkoxy, aryl, haloalkyl, haloalkoxy, -arylalkoxy, arylalkoxy-, cycloalkyl, —O-cycloalkyl, heterocyclyl, heteroaryl, alkylamino-, -alkyleneamine, alkoxyamino-, —O—CH₂-cycloalkyl; —COOR^{a}; —C(O)R^{b}; —C(S)R^{a}; —C(O)NR^{a}R^{b}; —NR^{a}C(O)NR^{b}R^{c}; —N(R^{a})SOR^{b}; —N(R^{a}) SO₂R^{b}; —NR^{a}C(O)OR^{b}; —NR^{a}R^{b}; —NR^{a}C(O)R^{b}; —NR^{a}C(S)R^{b}; —SONR^{a}R^{b}; —SO₂NR^{a}R^{b}; —OR^{a}; —OR^{a}C(O)OR^{b}; —OC(O)NR^{a}R^{b}; —OC(O)R^{a}; —R^{a}NR^{b}R^{c}; —R^{a}OR^{b}; —SR^{a}; —SOR^{a} and —SO₂R^{a};
each of R^{a}, R^{b} and R^{c} are independently a hydrogen atom or an optionally substituted alkyl, aryl, arylalkyl, cycloalkyl, heterocyclyl, heteroaryl or heteroarylalkyl;
wherein the substituents are further optionally substituted by one or more halogen, hydroxy, nitro, cycloalkyl, cyano, azido, nitroso, amino, hydrazino, formyl, alkyl, alkoxy, oxo, aryl, alkoxyamino- or haloalkyl;
wherein when the group R₁ is cyclic, R₁ is a residue formed by removing a hydrogen atom from a saturated or unsaturated monocyclic carbocyclic ring or a saturated or unsaturated monocyclic heterocyclic ring having one or two or three heteroatoms selected from a nitrogen atom, an oxygen atom and a sulfur atom in the ring, and a saturated or unsaturated monocyclic 3, 4, 5, 6, 7 or 8 membered heterocyclic ring having 3 to 5 carbon atoms, or a bicyclic or tricyclic condensed polycyclic heterocyclic ring having one or two or three heteroatoms in the ring, and a bicyclic or tricyclic condensed polycyclic heterocyclic ring having 7 to 13 carbon atoms, wherein the rings are optionally substituted with at least one substituent;
wherein R₂ is hydrogen or alkyl;
wherein R₃ is —NR^{d}R^{e};
each of R^{d} and R^{e} are independently selected from the group consisting of hydrogen or optionally substituted alkyl, alkenyl, aryl, arylalkyl, cycloalkyl, heterocyclyl, heteroaryl or heteroarylalkyl; or R^{d} and R^{e} together with the N, form an optionally substituted heterocyclic or heteroaryl ring;
wherein the substituents are further optionally substituted by one or more halogen, hydroxy, nitro, cycloalkyl, cyano, azido, nitroso, amino, hydrazino, formyl, alkyl, aryl, heterocyclyl, alkoxy, mercapto, alkylthio, alkylcarbonyl, carboxy, alkxoycarbonyl, alkoxycarbonyloxy, oxo, or haloalkyl;
wherein these further substituents are in turn further optionally substituted by one or more halogen, hydroxy, nitro, cycloalkyl, cyano, azido, nitroso, amino, hydrazino, formyl, alkyl, or haloalkyl;
their derivatives, analogs, tautomeric forms, stereoisomers, polymorphs, hydrates, solvates, intermediates, pharmaceutically acceptable salts, pharmaceutical compositions, metabolites and prodrugs thereof.

2. The compound of claim 1,
wherein R₁
represents hydrogen, optionally hydroxy-, amino-, carboxy-, C₁-C₆-alkyl-C₁-C₆ alkanoate-substituted C₁-C₂₀alkyl, C₂-C₂₀alkenyl, C₁-C₈-alkoxy-C₁-C₈-alkyl, or poly-C₁-C₈-alkoxy-C₁-C₈-alkyl, or represents mono-, bi-, or tricyclic C₃-C₁₃ cycloalkyl in which optionally one or two not directly adjacent methylene groups are replaced by oxygen and/or nitrogen, wherein cycloalkyl is optionally substituted with halogen, hydroxyl, amino, oxo, phenyl or C₁-C₈-alkoxy,
or represents phenyl-C₁-C₆-alkyl-, wherein phenyl is optionally substituted with one or more halogen, hydroxyl, amino, C₁-C₆-alkyl, C₁-C₆-alkoxy,
or represents optionally halogen- or C₁-C₆-alkyl-substituted 5- or 6-membered heteroaryl having one or two heteroatoms selected from the group consisting of oxygen and nitrogen,
or represents formula (IIa), (IIb), (IIc), (IId) or (IIe): 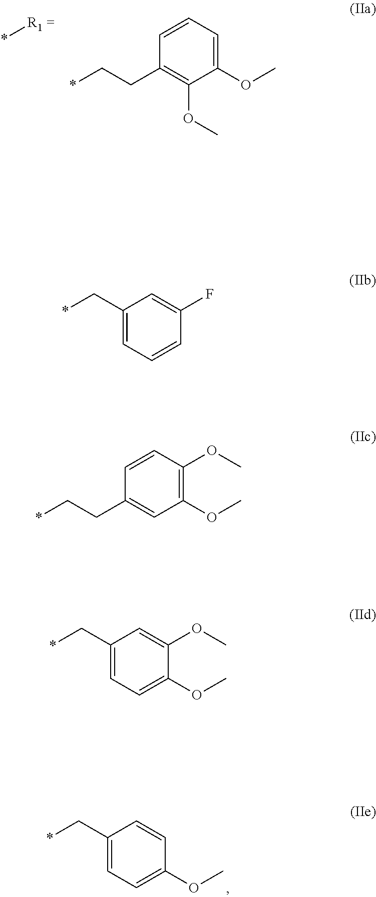
and
wherein R₂ represents hydrogen or C₁-C₆ alkyl.

3. The compound of claim 1,
wherein R₁R₂N is selected from the groups consisting of:

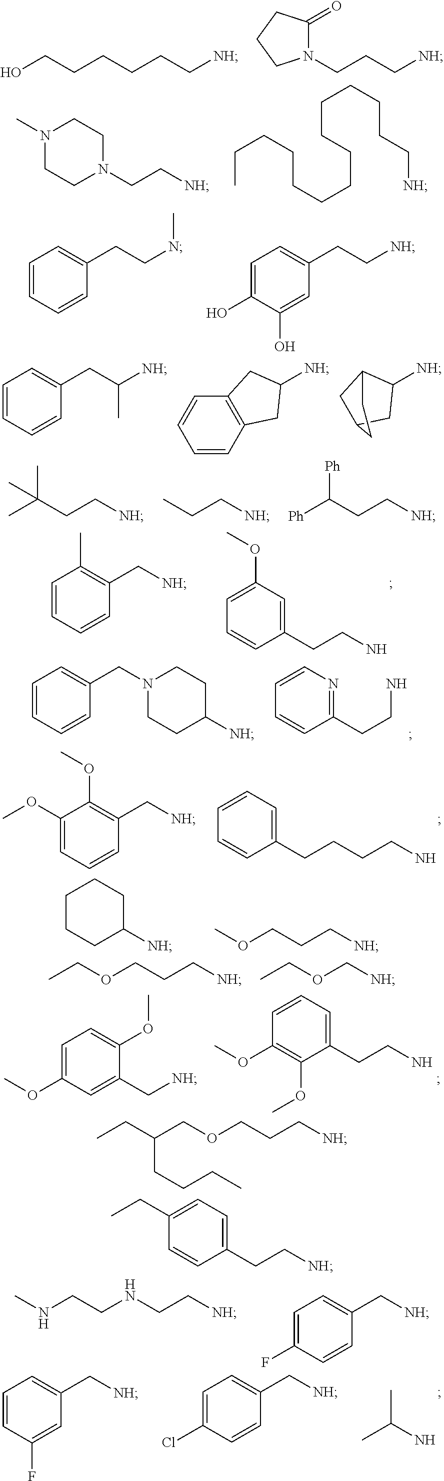

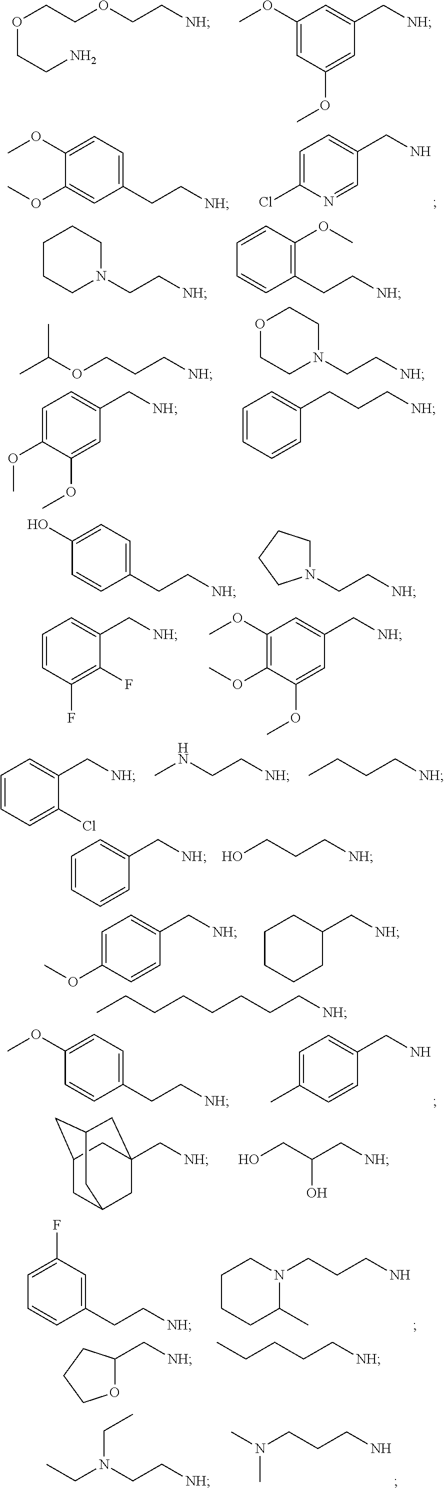

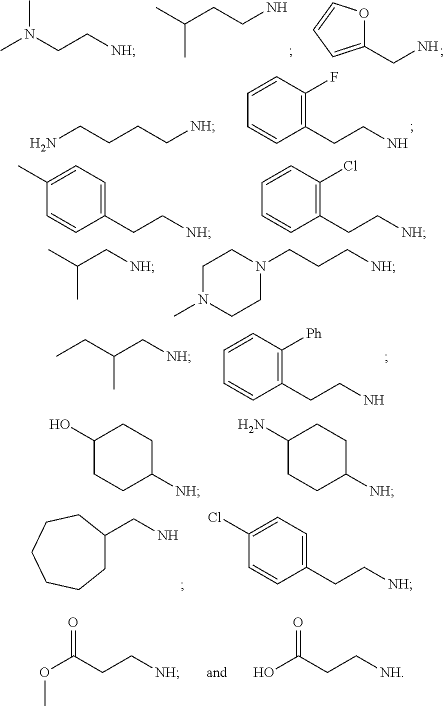

4. (canceled)

5. The compound according to claim 1, wherein R₃ represents the formula —NR^{d}R^{e},
wherein each of R^{d} and R^{e} is independently selected from the group consisting of a hydrogen atom, or an optionally substituted group selected from the group consisting of:
an alkyl group having 1 to 20 carbon atoms which may have one or two or three substituents selected from the group consisting of:
a halogen atom, a cycloalkyl group having 3 to 10 carbon atoms, an aryl group, a residue formed by removing a hydrogen atom from a saturated or unsaturated monocyclic heterocyclic ring having one or two or three heteroatoms selected from the group consisting of a nitrogen atom, an oxygen atom and a sulfur atom in the ring, a saturated or unsaturated monocyclic 5 or 6 membered heterocyclic ring having 3 to 5 carbon atoms, a bicyclic or tricyclic condensed polycyclic heterocyclic ring having one or two or three heteroatoms in the ring, a bicyclic or tricyclic condensed polycyclic heterocyclic ring having 7 to 13 carbon atoms group, and an alkoxy group having an alkyl moiety having 1 to 8 carbon atoms;
an alkenyl group having 2 to 8 carbon atoms;
a cycloalkyl group having 3 to 10 carbon atoms;
an aryl group having one or two or three substituents selected from the group consisting of:
a halogen atom, an alkyl group having 1 to 8 carbon atoms, a hydroxy group, an alkoxy group having an alkyl moiety having 1 to 8 carbon atoms, a mercapto group, an alkylthio group having an alkyl moiety having 1 to 8 carbon atoms, a formyl group, an alkylcarbonyl group having an alkyl moiety having 1 to 8 carbon atoms, a carboxy group, an alkoxycarbonyl group having an alkoxy group having an alkyl moiety having 1 to 8 carbon atoms, an alkylcarbonyloxy group having an alkylcarbonyl group having an alkyl moiety having 1 to 8 carbon atoms, a nitro group, a cyano group, a residue formed by removing a hydrogen atom from a saturated or unsaturated monocyclic heterocyclic ring having one or two or three heteroatoms selected from a nitrogen atom, an oxygen atom and a sulfur atom in the ring, and a saturated or unsaturated monocyclic 5 or 6 membered heterocyclic ring having 3 to 5 carbon atoms, or a bicyclic or tricyclic condensed polycyclic heterocyclic ring having one or two or three heteroatoms in the ring, and a bicyclic or tricyclic condensed polycyclic heterocyclic ring having 7 to 13 carbon atoms;
an optionally cyano-, hydroxy substituted C₁-C₂₀ alkyl;
an optionally halogen-, hydroxy-, amino-, oxo-, C₁-C₆-alkoxy substituted C₃-C₁₀ cycloalkyl, or C₁-C₆-alkoxy substituted C₃-C₁₀ heterocyclyl, wherein the heterocyclyl has one or two heteroatoms selected from the group consisting of nitrogen and/or oxygen;
a C₁-C₆-alkyl-C₁-C₆-alkanoate;
wherein the substituents in turn are further optionally substituted by one or more halogen, hydroxy, nitro, cycloalkyl, cyano, azido, nitroso, amino, hydrazino, formyl, alkyl, haloalkyl;
or wherein R₃ is represented by the formula 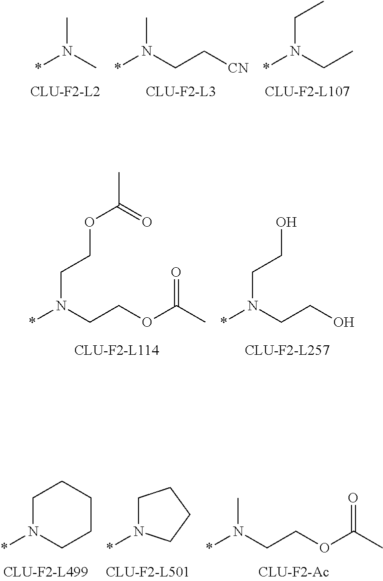
or their derivatives, analogs, tautomeric forms, stereoisomers, polymorphs, hydrates, solvates, intermediates, pharmaceutically acceptable salts, pharmaceutical compositions, metabolites and prodrugs thereof.

6. (canceled)

7. (canceled)

8. The compound of claim 1 having the formula selected from the group consisting of: 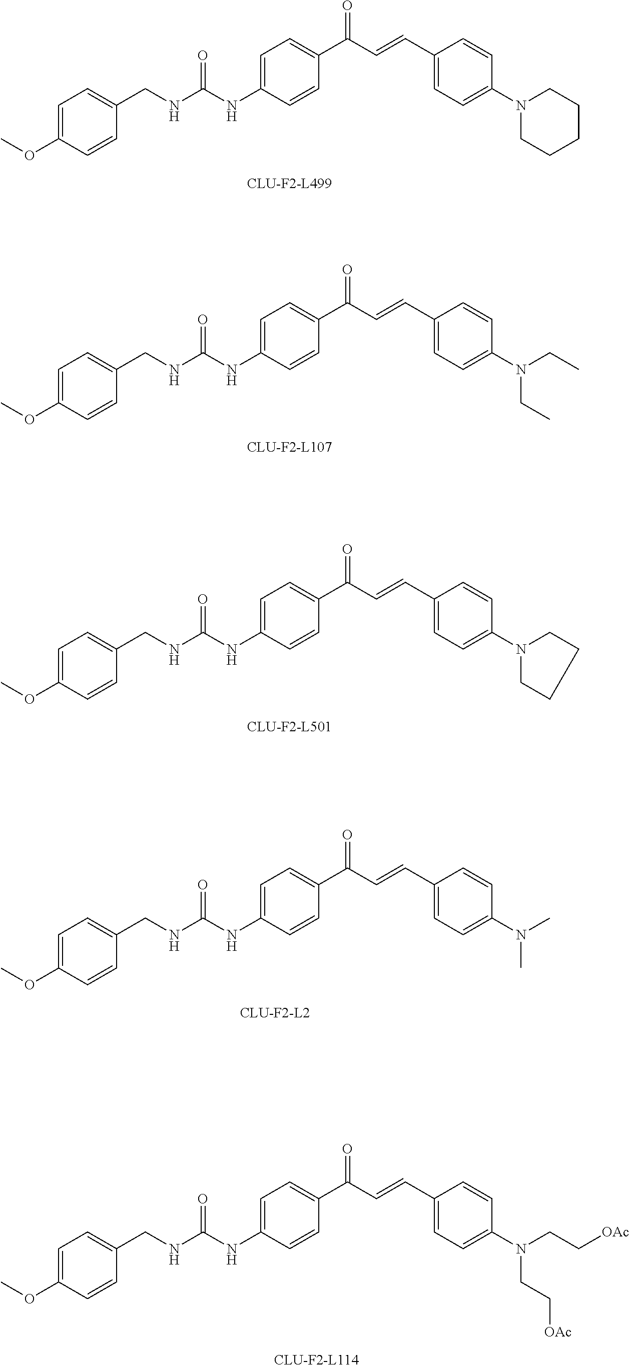 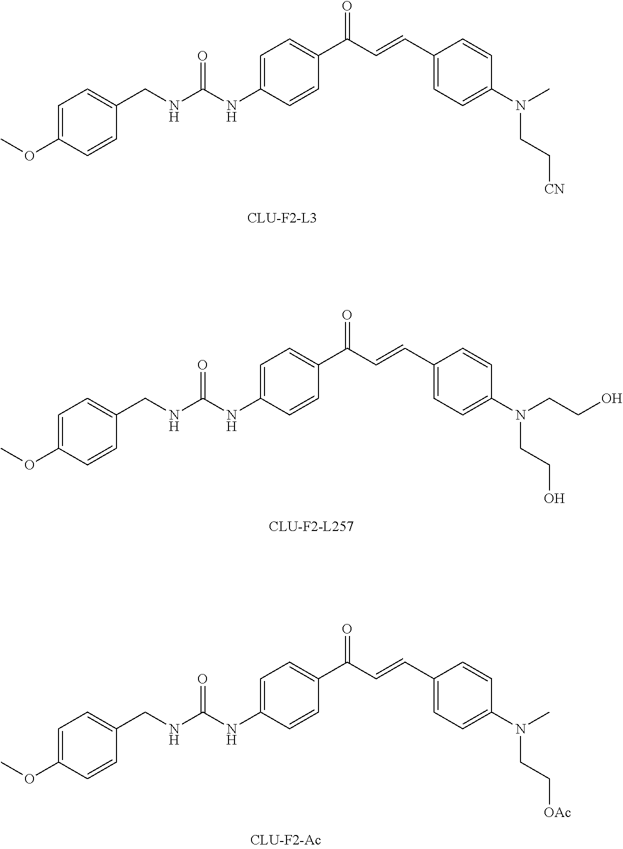 [SMILES: COc1ccc(CNC(=O)Nc2ccc(C(=O)/C=C/c3ccc(N(C)CCC#N)cc3)cc2)cc1 ; COc1ccc(CNC(=O)Nc2ccc(C(=O)/C=C/c3ccc(N(CCO)CCO)cc3)cc2)cc1 ; COc1ccc(CNC(=O)Nc2ccc(C(=O)/C=C/c3ccc(N(C)CCOC(C)=O)cc3)cc2)cc1]
their derivatives, analogs, tautomeric forms, stereoisomers, polymorphs, hydrates, solvates, intermediates, pharmaceutically acceptable salts, pharmaceutical compositions, metabolites and prodrugs thereof.

9. A method of detecting insulin, wherein the method comprises the use of the compound of claim 1.

10. (canceled)

11. (canceled)

12. The method according to claim 9, wherein insulin is to be detected in a sample selected from the group consisting of a cell culture, an isolated sample from a subject, a solution and an isolated cell from a tissue, preferably wherein detection comprises evaluating insulin secretion in the sample.

13. (canceled)

14. The method according to claim 9, wherein insulin is selected from the group consisting of human, bovine, murine and porcine insulin; or wherein insulin comprises the amino acid sequence selected from the group consisting of SEQ ID NO. 1 (GIVEQCCTSICSLYQLENYCN), SEQ ID NO. 2 (FVNQHLCGSHLVEALYLVCGERGFFYTPKT), SEQ ID NO. 3 (GIVEQCCASVCSLYQLENYCN), SEQ ID NO. 4 (FVNQHLCGSHLVEALYLVCGERGFFYTPKA), SEQ ID NO. 5 (GIVEQCCTSICSLYQLENYCN), SEQ ID NO. 6 (FVNQHLCGSHLVEALYLVCGERGFFYTPKA), SEQ ID NO. 7 (GIVDQCCTSICSLYQLENYCN) and SEQ ID NO. 8 (FVKQHLCGPHLVEALYLVCGERGFFYTPKS), wherein SEQ ID NOs. 1 to 8 is the amino acid sequence of the A and B chains of human, bovine, porcine and murine insulin, respectively; or wherein the insulin is encoded by a nucleic acid having the sequence selected from the group consisting of SEQ ID NO: 9 to 16:

| SEQ | |
|---|---|
| ID NO. | NUCLEIC ACID SEQUENCE |
| 9 | ggcattgtggaacagtgctgcaccagcatttgcagcct |
| | gtatcagctggaaaactattgcaac |
| 10 | tttgtgaaccagcatctgtgcggcagccatctggtgga |
| | agcgctgtatctggtgtgcggcgaacgcggcttttttt |
| | ataccccgaaaacc |
| 11 | ggcattgtggaacagtgctgcgcgagcgtgtgcagcct |
| | gtatcagctggaaaactattgcaac |
| 12 | tttgtgaaccagcatctgtgcggcagccatctggtgga |
| | agcgctgtatctggtgtgcggcgaacgcggcttttttt |
| | ataccccgaaagcg |
| 13 | ggcattgtggaacagtgctgcaccagcatttgcagcct |
| | gtatcagctggaaaactattgcaac |
| 14 | tttgtgaaccagcatctgtgcggcagccatctggtgga |
| | agcgctgtatctggtgtgcggcgaacgcggcttttttt |
| | ataccccgaaagcg |
| 15 | ggcattgtggatcagtgctgcaccagcatttgcagcct |
| | gtatcagctggaaaactattgcaac |
| 16 | tttgtgaaacagcatctgtgcggcccgcatctggtgga |
| | agcgctgtatctggtgtgcggcgaacgcggcttttttt |
| | ataccccgaaaagc. |

15. (canceled)

16. (canceled)

17. The method of claim 14, wherein the nucleic acid is comprised in a vector.

18. The method according to claim 9, wherein detecting insulin in a test sample comprises the steps of:
(a) contacting the test sample to be evaluated with a compound of formula (I): 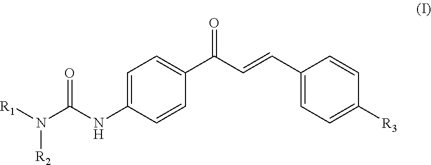
wherein the configuration around the double bond is E or Z;
wherein R₁ is selected from the group consisting of:
hydrogen,
cyclic aliphatic, acyclic aliphatic, branched aliphatic, unbranched aliphatic, substituted aliphatic, unsubstituted aliphatic,
cyclic heteroaliphatic, acyclic heteroaliphatic, branched heteroaliphatic, unbranched heteroaliphatic, substituted heteroaliphatic, unsubstituted heteroaliphatic,
substituted aryl, unsubstituted aryl,
substituted heteroaryl, unsubstituted heteroaryl,
cyclic acyl, acyclic acyl, substituted acyl and unsubstituted acyl;
wherein when the group R₁ is substituted, the one or more substituents are selected from the group consisting of halogen, hydroxyl, nitro, cyano, oxo (═O), thioxo (═S), azido, nitroso, amino, hydrazine, formyl, alkyl, alkene, alkyne, -alkyalkoxy, aryl, haloalkyl, haloalkoxy, -arylalkoxy, arylalkoxy-, cycloalkyl, —O-cycloalkyl, heterocyclyl, heteroaryl, alkylamino-, -alkyleneamine, alkoxyamino-, —O—CH₂-cycloalkyl; —COOR^{a}; —C(O)R^{b}; —C(S)R^{a}; —C(O)NR^{a}R^{b}; —NR^{a}C(O)NR^{b}R^{c}; —N(R^{a})SOR^{b}; —N(R^{a})SO₂R^{b}; —NR^{a}C(O)OR^{b}; —NR^{a}R^{b}; —NR^{a}C(O)R^{b}; —NR^{a}C(S)R^{b}; —SONR^{a}R^{b}; —SO₂NR^{a}R^{b}; —OR^{a}; —OR^{a}C(O)OR^{b}; —OC(O); —OC(O)R^{a}; —R^{a}NR^{b}R^{c}; —R^{a}OR^{b}; —SR^{a}; —SOR^{a} and —SO₂R^{a};
each of R^{a}, R^{b} and R^{c} are independently a hydrogen atom or an optionally substituted alkyl, aryl, arylalkyl, cycloalkyl, heterocyclyl, heteroaryl or heteroarylalkyl;
wherein the substituents are further optionally substituted by one or more halogen, hydroxy, nitro, cycloalkyl, cyano, azido, nitroso, amino, hydrazino, formyl, alkyl, alkoxy, oxo, aryl, alkoxyamino- or haloalkyl;
wherein when the group R₁ is cyclic, R₁ is a residue formed by removing a hydrogen atom from a saturated or unsaturated monocyclic carbocyclic ring or a saturated or unsaturated monocyclic heterocyclic ring having one or two or three heteroatoms selected from a nitrogen atom, an oxygen atom and a sulfur atom in the ring, and a saturated or unsaturated monocyclic 3, 4, 5, 6, 7 or 8 membered heterocyclic ring having 3 to 5 carbon atoms, or a bicyclic or tricyclic condensed polycyclic heterocyclic ring having one or two or three heteroatoms in the ring, and a bicyclic or tricyclic condensed polycyclic heterocyclic ring having 7 to 13 carbon atoms, wherein the rings are optionally substituted with at least one substituent;
wherein R₂ is hydrogen or alkyl;
wherein R₃ is —NR^{d}R^{e};
each of R^{d} and R^{e} are independently selected from the group consisting of hydrogen or optionally substituted alkyl, alkenyl, aryl, arylalkyl, cycloalkyl, heterocyclyl, heteroaryl or heteroarylalkyl; or R^{d} and R^{e} together with the N, form an optionally substituted heterocyclic or heteroaryl ring;
wherein the substituents are further optionally substituted by one or more halogen, hydroxy, nitro, cycloalkyl, cyano, azido, nitroso, amino, hydrazino, formyl, alkyl, aryl, heterocyclyl, alkoxy, mercapto, alkylthio, alkylcarbonyl, carboxy, alkxoycarbonyl, alkoxycarbonyloxy, oxo, or haloalkyl;
wherein these further substituents are in turn further optionally substituted by one or more halogen, hydroxy, nitro, cycloalkyl, cyano, azido, nitroso, amino, hydrazino, formyl, alkyl, or haloalkyl;
their derivatives, analogs, tautomeric forms, stereoisomers, polymorphs, hydrates, solvates, intermediates, pharmaceutically acceptable salts, pharmaceutical compositions, metabolites and prodrugs thereof;
(b) exciting the sample with a radiation source at a wavelength of about 430 nm, suitable to detect the compound;
(c) observing the sample in conjunction with means for detecting fluorescence intensity of the compound at an emission wavelength of about 560 nm;
(d) comparing the fluorescence in the test sample with the fluorescence in a control sample, wherein the control sample does not contain insulin;
wherein an increase in fluorescence intensity above a basal level of fluorescence in the control sample denotes the presence of insulin in the test sample.

19. The method of claim 18, wherein the compound is represented by the formula

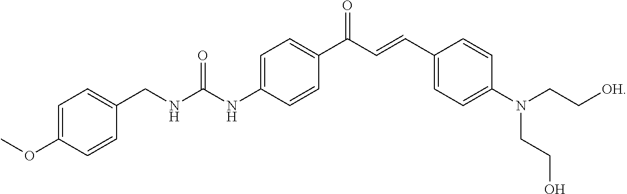 [SMILES: COc1ccc(CNC(=O)Nc2ccc(C(=O)/C=C/c3ccc(N(CCO)CCO)cc3)cc2)cc1]

20. The method according to claim 18, wherein detection comprises evaluating secretion of insulin by living cells, wherein said step of contacting the sample with the compound of formula (I) comprises incubating said cells in a medium containing said compound for sufficient time, such that said insulin is secreted in the medium where said insulin can be detected.

21. The method according to claim 20, wherein evaluating secretion comprises means for detecting fluorescence of the compound of formula (I) in real time, thereby providing real time monitoring of secretion of insulin by living cells.

22. A method for screening a compound that is suspected to affect secretion of insulin comprised in a test sample, comprising the steps of:
(a) contacting the compound to be screened with the test sample;
(b) contacting a compound of formula (I) with the test sample, wherein contacting is simultaneous, sequential or separate: 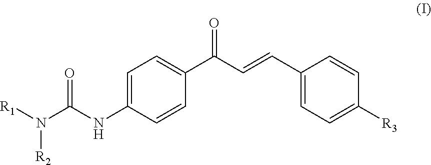
wherein the configuration around the double bond is E or Z;
wherein R₁ is selected from the group consisting of:
hydrogen,
cyclic aliphatic, acyclic aliphatic, branched aliphatic, unbranched aliphatic, substituted aliphatic, unsubstituted aliphatic,
cyclic heteroaliphatic, acyclic heteroaliphatic, branched heteroaliphatic, unbranched heteroaliphatic, substituted heteroaliphatic, unsubstituted heteroaliphatic,
substituted aryl, unsubstituted aryl,
substituted heteroaryl, unsubstituted heteroaryl,
cyclic acyl, acyclic acyl, substituted acyl and unsubstituted acyl;
wherein when the group R₁ is substituted, the one or more substituents are selected from the group consisting of halogen, hydroxyl, nitro, cyano, oxo (═O), thioxo (═S), azido, nitroso, amino, hydrazine, formyl, alkyl, alkene, alkyne, -alkyalkoxy, aryl, haloalkyl, haloalkoxy, -arylalkoxy, arylalkoxy-, cycloalkyl, —O-cycloalkyl, heterocyclyl, heteroaryl, alkylamino-, -alkyleneamine, alkoxyamino-, —O—CH₂-cycloalkyl; —COOR^{a}; —C(O)R^{b}; —C(S)R^{a}; —C(O)NR^{a}R^{b}; —NR^{a}C(O)NR^{b}R^{c}; —N(R^{a})SOR^{b}; —N(R^{a})SO₂R^{b}; —NR^{a}C(O)OR^{b}; —NR^{a}R^{b}; —NR^{a}C(O)R^{b}; —NR^{a}C(S)R^{b}; —SONR^{a}R^{b}; —SO₂NR^{a}R^{b}; —OR^{a}; —OR^{a}C(O)OR^{b}; —OC(O); —OC(O)R^{a}; —R^{a}NR^{b}R^{c}; —R^{a}OR^{b}; —SR^{a}; —SOR^{a} and —SO₂R^{a};
each of R^{a}, R^{b} and R^{c} are independently a hydrogen atom or an optionally substituted alkyl, aryl, arylalkyl, cycloalkyl, heterocyclyl, heteroaryl or heteroarylalkyl;
wherein the substituents are further optionally substituted by one or more halogen, hydroxy, nitro, cycloalkyl, cyano, azido, nitroso, amino, hydrazino, formyl, alkyl, alkoxy, oxo, aryl, alkoxyamino- or haloalkyl;
wherein when the group R₁ is cyclic, R₁ is a residue formed by removing a hydrogen atom from a saturated or unsaturated monocyclic carbocyclic ring or a saturated or unsaturated monocyclic heterocyclic ring having one or two or three heteroatoms selected from a nitrogen atom, an oxygen atom and a sulfur atom in the ring, and a saturated or unsaturated monocyclic 3, 4, 5, 6, 7 or 8 membered heterocyclic ring having 3 to 5 carbon atoms, or a bicyclic or tricyclic condensed polycyclic heterocyclic ring having one or two or three heteroatoms in the ring, and a bicyclic or tricyclic condensed polycyclic heterocyclic ring having 7 to 13 carbon atoms, wherein the rings are optionally substituted with at least one substituent;
wherein R₂ is hydrogen or alkyl;
wherein R₃ is —NR^{d}R^{e};
each of R^{d} and R^{e} are independently selected from the group consisting of hydrogen or optionally substituted alkyl, alkenyl, aryl, arylalkyl, cycloalkyl, heterocyclyl, heteroaryl or heteroarylalkyl; or R^{d} and R^{e} together with the N, form an optionally substituted heterocyclic or heteroaryl ring;
wherein the substituents are further optionally substituted by one or more halogen, hydroxy, nitro, cycloalkyl, cyano, azido, nitroso, amino, hydrazino, formyl, alkyl, aryl, heterocyclyl, alkoxy, mercapto, alkylthio, alkylcarbonyl, carboxy, alkxoycarbonyl, alkoxycarbonyloxy, oxo, or haloalkyl;
wherein these further substituents are in turn further optionally substituted by one or more halogen, hydroxy, nitro, cycloalkyl, cyano, azido, nitroso, amino, hydrazino, formyl, alkyl, or haloalkyl;
their derivatives, analogs, tautomeric forms, stereoisomers, polymorphs, hydrates, solvates, intermediates, pharmaceutically acceptable salts, pharmaceutical compositions, metabolites and prodrugs thereof;
(c) detecting a signal from the compound of formula (I), which is indicative of the presence of insulin in the test sample to obtain a first value; and
(d) comparing the first value with a control value, wherein the control value corresponds to a signal in a control sample, and the control value is determined from the control sample which is the same as provided in (a), except that the control sample is not contacted with the compound to be screened;
wherein if the test value is greater or smaller than the control value, then the compound affects secretion of insulin in the sample.

23. The method of claim 22, wherein the test sample is selected from the group consisting of a cell culture, an isolated sample from a subject and an isolated islet; or wherein the compound to be screened is selected from the group consisting of KCl, glucose, Forskolin ((3R,4aR,5S, 6S, 6aS, 10S, 10aR, 10bS)-6, 10, 10b-trihydroxy-3, 4a, 7, 7, 10a-pentamethyl-1-oxo-3-vinyldodecahydro-1H-benzo[f]chromen-5-yl acetate), 3-isobutyl-1-methylxanthine (IBMX; 1-methyl-3-(2-methylpropyl)-7H-purine-2,6-dione), Glibenclamide (5-chloro-N-(4-[N-(cyclohexylcarbamoyl)sulfamoyl]phenethyl)-2-methoxybenzamide), amiodarone ((2-{4-[(2-butyl-1-benzofuran-3-yl)carbonyl]-2,6-diiodophenoxy}ethyl)diethylamine), nifedipine (3,5-dimethyl 2,6-dimethyl-4-(2-nitrophenyl)-1,4-dihydropyridine-3,5-dicarboxylate), Leucine, Tolbutamide (N-[(butylamino)carbonyl]-4-methylbenzenesulfonamide), Ouabain (1β,3β, 5β, 11α, 14, 19-Hexahydroxycard-20 (22)-enolide 3-(6-deoxy-α-L-mannopyranoside), 4-[(1R,3S,5S, 8R, 9S, 10R, 11R, 13R, 14S, 17R)-1,5,11,14-tetrahydroxy-10-(hydroxymethyl)-13-methyl-3-((2R,3R,4R,5R,6S)-3,4,5-trihydroxy-6-methyltetrahydro-2H-pyran-2-yloxy)hexadecahydro-1H-cyclopenta[a]phenanthren-17-yl]furan-2 (5H)-one) and adrenalin.

24. The method of claim 22, wherein the contacting as provided in step (b) is done simultaneously, separately, or sequentially; or wherein detecting as provided in step (c) is real time detection.

25.-28. (canceled)

29. A method of assessing whether a condition or a stimulus affects secretion of insulin in cultured cells, comprising:
(a) culturing cells expressing insulin under a condition or stimulus to be assessed for its effect on secretion of insulin, wherein the cells are cultured in the presence of a compound of formula (I), wherein the cells are referred to as test cells: 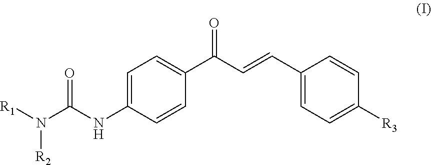
wherein the configuration around the double bond is E or Z;
wherein R₁ is selected from the group consisting of:
hydrogen,
cyclic aliphatic, acyclic aliphatic, branched aliphatic, unbranched aliphatic, substituted aliphatic, unsubstituted aliphatic,
cyclic heteroaliphatic, acyclic heteroaliphatic, branched heteroaliphatic, unbranched heteroaliphatic, substituted heteroaliphatic, unsubstituted heteroaliphatic,
substituted aryl, unsubstituted aryl,
substituted heteroaryl, unsubstituted heteroaryl,
cyclic acyl, acyclic acyl, substituted acyl and unsubstituted acyl;
wherein when the group R₁ is substituted, the one or more substituents are selected from the group consisting of halogen, hydroxyl, nitro, cyano, oxo (═O), thioxo (═S), azido, nitroso, amino, hydrazine, formyl, alkyl, alkene, alkyne, -alkyalkoxy, aryl, haloalkyl, haloalkoxy, -arylalkoxy, arylalkoxy-, cycloalkyl, —O-cycloalkyl, heterocyclyl, heteroaryl, alkylamino-, -alkyleneamine, alkoxyamino-, —O—CH₂-cycloalkyl; —COOR^{a}; —C(O)R^{b}; —C(S)R^{a}; —C(O)NR^{a}R^{b}; —NR^{a}C(O)NR^{b}R^{c}; —N(R^{a})SOR^{b}; —N(R^{a})SO₂R^{b}; —NR^{a}C(O)OR^{b}; —NR^{a}R^{b}; —NR^{a}C(O)R^{b}; —NR^{a}C(S)R^{b}; —SONR^{a}R^{b}; —SO₂NR^{a}R^{b}; —OR^{a}; —OR^{a}C(O)OR^{b}; —OC(O); —OC(O)R^{a}; —R^{a}NR^{b}R^{c}; —R^{a}OR^{b}; —SR^{a}; —SOR^{a} and —SO₂R^{a};
each of R^{a}, R^{b} and R^{c} are independently a hydrogen atom or an optionally substituted alkyl, aryl, arylalkyl, cycloalkyl, heterocyclyl, heteroaryl or heteroarylalkyl;
wherein the substituents are further optionally substituted by one or more halogen, hydroxy, nitro, cycloalkyl, cyano, azido, nitroso, amino, hydrazino, formyl, alkyl, alkoxy, oxo, aryl, alkoxyamino- or haloalkyl;
wherein when the group R₁ is cyclic, R₁ is a residue formed by removing a hydrogen atom from a saturated or unsaturated monocyclic carbocyclic ring or a saturated or unsaturated monocyclic heterocyclic ring having one or two or three heteroatoms selected from a nitrogen atom, an oxygen atom and a sulfur atom in the ring, and a saturated or unsaturated monocyclic 3, 4, 5, 6, 7 or 8 membered heterocyclic ring having 3 to 5 carbon atoms, or a bicyclic or tricyclic condensed polycyclic heterocyclic ring having one or two or three heteroatoms in the ring, and a bicyclic or tricyclic condensed polycyclic heterocyclic ring having 7 to 13 carbon atoms, wherein the rings are optionally substituted with at least one substituent;
wherein R₂ is hydrogen or alkyl;
wherein R₃ is —NR^{d}R^{e};
each of R^{d} and R^{e} are independently selected from the group consisting of hydrogen or optionally substituted alkyl, alkenyl, aryl, arylalkyl, cycloalkyl, heterocyclyl, heteroaryl or heteroarylalkyl; or R^{d} and R^{e} together with the N, form an optionally substituted heterocyclic or heteroaryl ring;
wherein the substituents are further optionally substituted by one or more halogen, hydroxy, nitro, cycloalkyl, cyano, azido, nitroso, amino, hydrazino, formyl, alkyl, aryl, heterocyclyl, alkoxy, mercapto, alkylthio, alkylcarbonyl, carboxy, alkxoycarbonyl, alkoxycarbonyloxy, oxo, or haloalkyl;
wherein these further substituents are in turn further optionally substituted by one or more halogen, hydroxy, nitro, cycloalkyl, cyano, azido, nitroso, amino, hydrazino, formyl, alkyl, or haloalkyl;
their derivatives, analogs, tautomeric forms, stereoisomers, polymorphs, hydrates, solvates, intermediates, pharmaceutically acceptable salts, pharmaceutical compositions, metabolites and prodrugs thereof;
(b) detecting a signal from the compound, which is indicative of the presence of insulin in an extracellular environment of the test cells to obtain a first value;
(c) comparing the first value with a control value, wherein the control value corresponds to a signal in the extracellular environment of the control cells, and the control value is determined from control cells which are the same cells as cultured in (a), and which are cultured under the same conditions as in (a), except that the control cells are not cultured under the condition or stimulus to be assessed,
wherein if the test value is smaller or greater than the control value, then the condition or stimulus affects secretion of insulin in the cells.

30. The method of claim 29, wherein the condition or stimulus to be assessed is selected from the group consisting of one or more of pH, temperature, pressure, medium composition comprising serum, salts, vitamins, hormones, proteins, carbohydrates, lipids and chemical compounds, age and origin of the culture, atmosphere composition comprising CO₂ and O₂ concentration, cell culture support comprising two-dimensional culture, three-dimensional scaffold, and suspension culture.

31. The method of claim 29, wherein detecting as provided in step (c) is real time detection.

32. The method of claim 29, wherein the compound is represented by a formula 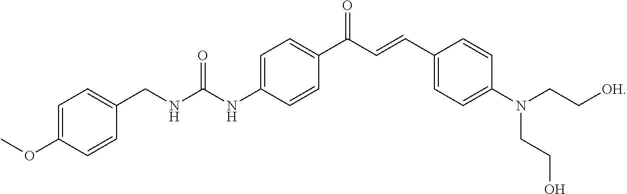 [SMILES: COc1ccc(CNC(=O)Nc2ccc(C(=O)/C=C/c3ccc(N(CCO)CCO)cc3)cc2)cc1]
